(19) United States
(12) Patent Application Publication
(10) Pub. No.: US 2022/0073495 A1
(43) Pub. Date: Mar. 10, 2022

(54) METHODS AND COMPOSITIONS FOR THE TREATMENT OF INFLUENZA

(71) Applicant: The Board of Regents of The Universtiy of Texas System, Austin, TX (US)

(72) Inventors: Beatriz M.A. Fontoura, Dallas, TX (US); Matthew A. Esparza, Dallas, TX (US); Hanspeter Niederstrasser, Coppell, TX (US); Bruce Posner, Richardson, TX (US); Joseph M. Ready, Carrollton, TX (US); Amir Mor, Nes-Ziona (IL)

(73) Assignee: The Board of Regents of The Universtiy of Texas System, Austin, TX (US)

(21) Appl. No.: 17/527,907

(22) Filed: Nov. 16, 2021

Related U.S. Application Data
(63) Continuation of application No. PCT/US2021/070286, filed on Mar. 18, 2021.
(60) Provisional application No. 62/991,445, filed on Mar. 18, 2020.

(51) Int. Cl. C07D 401/12; C07D 235/28; C07D 413/12

(52) U.S. Cl. CPC C07D 401/12; C07D 413/12; C07D 235/28

(57) ABSTRACT

The present disclosure provides, in part, compositions, kits and methods for treating or preventing an influenza viral infection by administering a therapeutically effective amount of an inhibitor of influenza viral M mRNA nuclear export to a subject in need.

### CROSS REFERENCE TO RELATED APPLICATIONS

This application is a continuation of International Patent Application Ser. No. PCT/US2021/070286 filed Mar. 18, 2021, which claims the benefit of U.S. Provisional Patent Application Ser. No. 62/991,445 filed Mar. 18, 2020, the contents of which are hereby incorporated by reference in their entireties.

### ACKNOWLEDGEMENT OF GOVERNMENT SUPPORT

This invention was made with government support under Grant Number AI119304 awarded by the National Institutes of Health. The government has certain rights in the invention.

### STATEMENT REGARDING SEQUENCE LISTING

The instant application contains a Sequence Listing which has been submitted via ASCII copy created on Mar. 18, 2021, referred to as ‘106546-698161_Seq_Listing_ST25.txt’ having 75 sequences.

### FIELD

The present disclosure generally relates to influenza treatment or prevention. In particular, the present disclosure relates to compounds that prevent influenza virus replication through inhibition of influenza viral mRNA nuclear export, as well as methods for treating or preventing influenza using the compounds.

### BACKGROUND

Influenza virus is a major human pathogen that kills approximately 500,000 people worldwide every year and between 15,000 to 40,000 Americans yearly, depending on the influenza strain. The 1918 influenza pandemic killed approximately 50 million people worldwide. Currently available prevention measures and treatments include vaccines and a few antiviral drugs. However, these treatment methods are limited by the mutability of the virus and the development of resistance. As antiviral treatments currently approved for clinical use target viral proteins directly, such treatments have an increased probability of developing strains resistant to these antiviral compositions.

Additionally, antiviral drugs are largely only effective if administered in the first 48 hours following infection and vaccines are less effective in treating elderly populations. Accordingly, in view of the lack of robust and diverse medical interventions available, additional antiviral compositions, methods, and therapeutic strategies for the treatment or prevention of influenza are desirable. The present disclosure fulfills this long standing need.

### SUMMARY

The present disclosure provides, in part, identification of compounds that inhibit influenza viral M mRNA processing and nuclear export and are useful in treatment or prevention of an influenza viral infection. The identified compounds target cellular proteins instead of viral proteins. The present disclosure further provides methods of treating or preventing an influenza viral infection using the compounds identified herein and kits comprising the same.

Accordingly, one aspect of the present disclosure provides a method of treating an influenza viral infection in a subject in need thereof. Such method comprises administering to the subject a therapeutically effective amount of an inhibitor of influenza viral M mRNA nuclear export. In one embodiment, the inhibitor targets a cellular protein in viral M mRNA speckle-export pathway. By way of non-limiting example, the cellular protein is a binding partner of viral NS1 protein.

In one embodiment, the inhibitor is a compound comprising a structural formula selected from the group consisting of Structural Formula I, Structural Formula II, Structural Formula III, Structural Formula IV, Structural Formula V, Structural Formula VI, Structural Formula VII, Structural Formula VIII, Structural Formula IX, Structural Formula X, Structural Formula XI, Structural Formula XII, Structural Formula XIII, Structural Formula XIV, Structural Formula XV, Structural Formula XVI, Structural Formula XVII, Structural Formula XVIII, Structural Formula XIX, Structural Formula XX, Structural Formula XXI, Structural Formula XXII, Structural Formula XXIII, Structural Formula XIV, Structural Formula XV, Structural Formula XVI, Structural Formula XVII, Structural Formula XVIII, and Structural Formula XXIX, or a pharmaceutically acceptable salt, solvate, hydrate, prodrug, or derivative thereof.

In one embodiment, the inhibitor is a compound comprising Structural Formula I below:

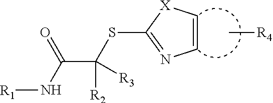

or a pharmaceutically acceptable salt, solvate, hydrate, prodrug, or derivative thereof; wherein R₁ is an unsubstituted or substituted aryl or heteroaryl; R₂ and R₃ are either the same or different and are selected from H or alkyl; X is selected from NH, NR₅, O, and S; R₄ is appended to an optional ring as part of a benzo-fused heteroaryl and is selected from H, alkyl or halogen; and R5 is an alkyl or aryl.

In one embodiment, the inhibitor is a compound comprising Structural Formula II below:

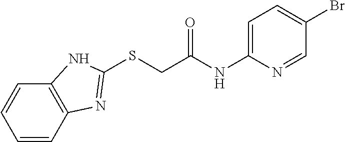 [SMILES: O=C(CSc1nc2ccccc2[nH]1)Nc1ccc(Br)cn1]

or a pharmaceutically acceptable salt, solvate, hydrate, prodrug, or derivative thereof.

In one embodiment, the inhibitor is a compound comprising Structural Formula III below:

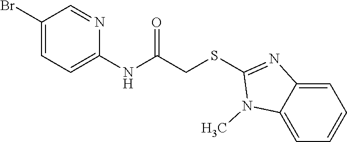 [SMILES: Cn1c(SCC(=O)Nc2ccc(Br)cn2)nc2ccccc21]

or a pharmaceutically acceptable salt, solvate, hydrate, prodrug, or derivative thereof.

In one embodiment, the inhibitor is a compound comprising Structural Formula IV below:

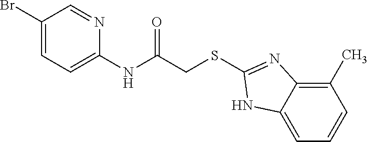 [SMILES: Cc1cccc2[nH]c(SCC(=O)Nc3ccc(Br)cn3)nc12]

or a pharmaceutically acceptable salt, solvate, hydrate, prodrug, or derivative thereof.

Another aspect of the present disclosure provides a method of preventing an influenza viral infection in a subject in need thereof. Such method comprises administering to the subject a therapeutically effective amount of an inhibitor of influenza viral M mRNA nuclear export. In one embodiment, the inhibitor targets a cellular protein in viral M mRNA speckle-export pathway. By way of non-limiting example, the cellular protein is a binding partner of viral NS1 protein.

In one embodiment, the inhibitor is a compound comprising a structural formula selected from the group consisting of Structural Formula I, Structural Formula II, Structural Formula III, Structural Formula IV, Structural Formula V, Structural Formula VI, Structural Formula VII, Structural Formula VIII, Structural Formula IX, Structural Formula X, Structural Formula XI, Structural Formula XII, Structural Formula XIII, Structural Formula XIV, Structural Formula XV, Structural Formula XVI, Structural Formula XVII, Structural Formula XVIII, Structural Formula XIX, Structural Formula XX, Structural Formula XXI, Structural Formula XXII, Structural Formula XXIII, Structural Formula XIV, Structural Formula XV, Structural Formula XVI, Structural Formula XVII, Structural Formula XVIII, and Structural Formula XXIX, or a pharmaceutically acceptable salt, solvate, hydrate, prodrug, or derivative thereof.

In one embodiment, the inhibitor is a compound comprising Structural Formula I below:

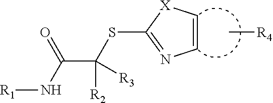

or a pharmaceutically acceptable salt, solvate, hydrate, prodrug, or derivative thereof; wherein R₁ is an unsubstituted or substituted aryl or heteroaryl; R₂ and R₃ are either the same or different and are selected from H or alkyl; X is selected from NH, NR₅, O, and S; R₄ is appended to an optional ring as part of a benzo-fused heteroaryl and is selected from H, alkyl or halogen; and R5 is an alkyl or aryl.

In one embodiment, the inhibitor is a compound comprising Structural Formula II below:

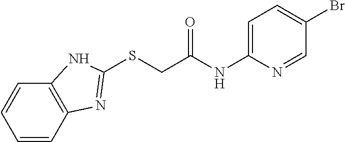 [SMILES: O=C(CSc1nc2ccccc2[nH]1)Nc1ccc(Br)cn1]

or a pharmaceutically acceptable salt, solvate, hydrate, prodrug, or derivative thereof.

In one embodiment, the inhibitor is a compound comprising Structural Formula III below:

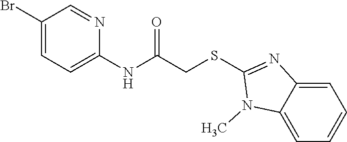 [SMILES: Cn1c(SCC(=O)Nc2ccc(Br)cn2)nc2ccccc21]

or a pharmaceutically acceptable salt, solvate, hydrate, prodrug, or derivative thereof.

In one embodiment, the inhibitor is a compound comprising Structural Formula IV below:

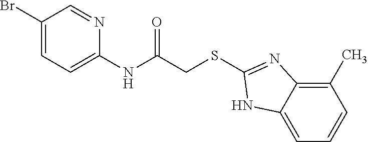 [SMILES: Cc1cccc2[nH]c(SCC(=O)Nc3ccc(Br)cn3)nc12]

or a pharmaceutically acceptable salt, solvate, hydrate, prodrug, or derivative thereof.

Still another aspect of the present disclosure provides a kit for treating or preventing an influenza viral infection. Such kit comprises a therapeutically effective amount of an inhibitor of influenza viral M mRNA nuclear export, a means of administering the inhibitor, and instructions for use.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to describe the manner in which the advantages and features of the disclosure can be obtained, reference is made to embodiments thereof which are illustrated in the appended drawings. It is to be understood that these drawings depict only exemplary embodiments of the disclosure and are not therefore to be considered to be limiting of its scope, the principles herein are described and explained with additional specificity and detail through the use of the accompanying drawings.

FIG. 1 depicts disruption of the NS1-BP gene by CRISPR-Cas9 system yielded A549 cells lacking NS1-BP protein. In particular, cell lysates from control or NS1-BP knockout cells were subjected to western blot analysis with antibodies against NS1-BP antibody or β-tubulin, as control.

FIG. 2 depicts wild-type or NS1-BP^{-/-} A549 cells were infected with influenza virus (A/WSN/33) at MOI 2 for 6 h. In particular, single-molecule RNA fluorescence in situ hybridization (smFISH) was performed to detect influenza virus M mRNA. Hoechst staining labeled nuclei. Scale bar=10 μm.

FIG. 3 depicts quantification of total fluorescence intensity of M mRNA in the nucleus and cytoplasm of wild-type or NS1-BP^{-/-} cells from FIG. 2.

FIG. 4 depicts quantification of nuclear-to-cytoplasmic (N/C) ratios of M mRNA in wild-type or NS1-BP^{-/-} cells from FIG. 2.

FIG. 5 depicts non-infected wild-type or NS1-BP^{-/-} A549 cells were subjected to RNA-FISH to label poly(A) RNA.

FIG. 6 depicts quantification of total fluorescence intensity of poly(A) RNA in the nucleus and cytoplasm of wild-type or NS1-BP^{-/-} cells from FIG. 5. Thirty cells were quantified for each condition. These data are representative of three independent experiments. ***p<0.001.

FIG. 7 depicts quantification of nuclear-to-cytoplasmic (N/C) ratios of poly(A) RNA in the wild-type or NS1-BP^{-/-} cells from FIG. 5. Thirty cells were quantified for each condition. These data are representative of three independent experiments. ***p<0.001.

FIG. 8 depicts a schematic representation of a high-throughput screen to identify chemical inhibitors of viral M mRNA processing and nuclear export. In particular, a screen was performed using a chemical library of 232,500 compounds in A549 cells. Cells were incubated with compounds for 30 min and, for robust imaging analysis, ˜100% of the cells were infected with influenza virus (WSN), at MOI 2 for 7.5 h. Viral M mRNA was detected by smRNA-FISH and images were systematically taken in a high throughput microscope (IN Cell Analyzer 6000). Samples on 384-well black clear-bottom plates were imaged at 20× magnification using the Hoechst and dsRed filters. Four fields of view per well were collected for each channel. The distribution of fluorescent signal between the nucleus (N) and the cytoplasm (N/C ratio) as well as total cell signal intensity were quantified using GE IN Cell Analyzer Workstation (version 3.7.3) and Pipeline Pilot (version 9.5; Biovia). Data was imported into the GeneData's Screener™ software analysis suite for quality control to ensure that data quality is high for all plates in each experimental run (Z′>0.4).

FIG. 9 depicts representative images showing uninfected cells; cells infected with A/WSN/33 and pretreated with 0.5 DMSO (control), which show viral M mRNA exported into the cytoplasm, in red; and cells infected with A/WSN/33 pre-treated with 2.5 μM of the transcription inhibitor DRB (5,6-dichloro-1-β-D-ribofuranosylbenzimidazole). DRB served as positive control for viral M mRNA nuclear retention.

FIG. 10 depicts distribution of nuclear (N) to cytosolic (C) (N/C ratio) of all wells in a mock assay plate showing the assay window and sensitivity. DMSO wells were normalized to 0 and DRB positive control wells were normalized to 100. The circled red diamond shape represents a lower dose of DRB and shows lower normalized N/C ratio than the other diamonds representing the full control dose of DRB.

FIG. 11 depicts rank-sorted Z-score of the Nuclear to Cytoplasmic (N/C) ratio of viral M mRNA in A549 cells after individual treatment with 232,500 compounds (2.5 μM). Each N/C value is expressed as a Z-score, indicating the number of standard deviations from the median plate ratio. Points above the red line at Z-score 3 represent compounds that were considered hits in the primary screen.

FIG. 12 depicts rank-sorted Z-score of the total intensity of viral M mRNA after compound treatment. Each value is expressed as a Z-score, indicating the number of standard deviations from the median plate intensity. Points below the red line at Z-score −3 represent compounds selected as hits in the primary screen. To better visualize the distribution of compounds within the desired range (Z-score<−3), the Z-score range of the graph has been focused to view data points that show decrease in viral M mRNA fluorescence.

FIG. 13 depicts a schematic representation of identification and selection of top hits that inhibit viral M mRNA nuclear export and/or expression. Out of the 232,500 compounds tested in the primary screen, compounds with Z-scores≥3 for the N/C ratio and compounds that decreased viral mRNA levels with Z-scores≤−3 were selected. Compounds that reduced nuclear count significantly (Z-score<−3) were considered cytotoxic and were eliminated from further consideration. Of those remaining, the 1,125 compounds with the highest Z-scores were chosen for confirmation studies. The top 600 compounds from single-dose confirmation studies were further evaluated in a 12-point dose response study to assess the potency (AC50—concentration at 50% activity). Examples of dose-response curves showing phenotypes of hits that induced viral M mRNA nuclear export block (increased N/C) and decrease in viral M mRNA levels (decreased intensity) are depicted. During this step, bulk cellular poly(A) RNA localization and intensity were also assessed by smRNA-FISH to determine the effect of these compounds on host cell mRNA (intensity and N/C ratio). Compounds that inhibited viral mRNA nuclear export and/or decreased viral mRNA levels but had no significant effect on the host cell poly(A) RNA were then selected for additional assays.

FIG. 14 depicts RNA-FISH and smRNA-FISH followed by fluorescence microscopy were performed in cells treated with 0.1% DMSO or 2.5 μM compound 2 to detect poly(A) RNA and GAPDH mRNA respectively, in uninfected cells.

FIG. 15 depicts total fluorescence intensity or nuclear to cytoplasmic fluorescence intensity (N/C ratio) quantified for poly(A) RNA and GAPDH mRNA in the absence or presence of compound 2 for (C, n=174 cells; Compound 2, n=181).

FIG. 16 depicts total fluorescence intensity or nuclear to cytoplasmic fluorescence intensity (N/C ratio) quantified for poly(A) RNA and GAPDH mRNA in the absence or presence of compound 2 for (C, n=172 cells; Compound 2, n=181 cells).

FIG. 17 depicts total fluorescence intensity or nuclear to cytoplasmic fluorescence intensity (N/C ratio) quantified for poly(A) RNA and GAPDH mRNA in the absence or presence of compound 2 for (C, n=166 cells; Compound 2, n=181 cells).

FIG. 18 depicts total fluorescence intensity or nuclear to cytoplasmic fluorescence intensity (N/C ratio) quantified for poly(A) RNA and GAPDH mRNA in the absence or presence of compound 2 for (C, n=151 cells; Compound 2, n=160 cells).

FIG. 19 depicts cells treated as in FIG. 14 except that smRNA-FISH was performed with probes to detect M mRNA in cells infected with WSN at MOI 2 for 8 h.

FIG. 20 depicts total fluorescence intensity or nuclear to cytoplasmic fluorescence intensity (N/C ratio) quantified for M mRNA in the absence or presence of compound 2 for (C, n=91 cells; Compound 2, n=104 cells).

FIG. 21 depicts total fluorescence intensity or nuclear to cytoplasmic fluorescence intensity (N/C ratio) quantified for M mRNA in the absence or presence of compound 2 for (C, n=101 cells; Compound 2, n=95 cells).

FIG. 22 depicts cells treated as in FIG. 19 except that smRNA-FISH was performed with probes to detect HA mRNA.

FIG. 23 depicts total fluorescence intensity or nuclear to cytoplasmic fluorescence intensity (N/C ratio) quantified for HA mRNA in the absence or presence of compound 2 for (C, n=104 cells; Compound 2, n=137 cells).

FIG. 24 depicts total fluorescence intensity or nuclear to cytoplasmic fluorescence intensity (N/C ratio) quantified for HA mRNA in the absence or presence of compound 2 for (C, n=101 cells; Compound 2, n=126 cells).

FIG. 25 depicts cells treated as in FIG. 19 except that smRNA-FISH was performed with probes to detect NS mRNA.

FIG. 26 depicts total fluorescence intensity or nuclear to cytoplasmic fluorescence intensity (N/C ratio) were quantified for M mRNA in the absence or presence of compound 2 for (C, n=96 cells; Compound 2, n=135 cells).

FIG. 27 depicts total fluorescence intensity or nuclear to cytoplasmic fluorescence intensity (N/C ratio) were quantified for M mRNA in the absence or presence of compound 2 for (C, n=106 cells; Compound 2, n=113 cells). At least three independent experiments were performed for each imaging analysis.

FIG. 28 depicts relative mRNA ratios of M2 to M1 determined by qPCR from RNA obtained from cells infected as in FIG. 19 and treated with 0.1% DMSO, 1 μM, or 2.5 μM compound 2. The nuclear speckle assembly factor SON was knocked down with siRNAs as positive control for inhibition of M1 to M2 mRNA splicing. Three independent experiments were performed. C, control.

FIG. 29 depicts relative mRNA ratios of NS2 to NS1 determined by qPCR from RNA obtained from cells infected as in FIG. 19 and treated with 0.1% DMSO, 1 μM, or 2.5 μM compound 2. The nuclear speckle assembly factor SON was knocked down with siRNAs as positive control for inhibition of M1 to M2 mRNA splicing. Three independent experiments were performed. C, control.

FIG. 30 depicts cellular ATP levels measured in cells treated with 0.1% DMSO or 2.5 μM of compound 2 at 24 h. Four independent experiments were performed and each contained 6 technical replicates. Graphs shows data points and mean +/−SD. *p<0.05; ***p<0.001 p<0.0001.

FIG. 31 depicts partial depletion of the mRNA export factor UAP56 show differential export of viral mRNAs similar to compound 2. (A) smRNA-FISH followed by fluorescence microscopy was performed to detect M mRNA in A549 cells treated with control siRNA or with two concentrations (25 nM and 50 nM) of siRNAs that target the coding region of UAP56 or control siRNA followed by infection with WSN at MOI 2 for 8 h. (B) Total fluorescence intensity or nuclear to cytoplasmic fluorescence intensity (N/C ratio) (C) were quantified for images in A in which cells were treated with 25 nM siRNA targeting UAP56. For B (C, n=117 cells; siRNA UAP56, n=171 cells) and C (Control, n=97 cells; siRNA UAP56, n=166 cells). Graphs show data points and mean +/- SD. ****p<0.0001. (D) A549 cells were treated with 25 nM siRNA targeting UAP56 or control siRNA as in A. RNA-FISH was performed to detect poly(A) RNA. Total fluorescence intensity (E) or nuclear to cytoplasmic fluorescence intensity (F) were quantified for images in D. For (E) (C, n=171 cells; siRNA UAP56, n=213 cells) and F (Control, n=172 cells; siRNA UAP56, n=208 cells). Graphs show data points and mean +/−SD. ****p<0.0001. (G-J) A549 cells were treated with 1 nM or 20 nM siRNA targeting the 3′UTR of the UAP56 mRNA or with control siRNA and then infected with WSN at MOI 2 for 8 h. (G) Purified RNA from total cell lysates was subjected to qPCR to measure UAP56 mRNA levels. (H) Cell lysates were also subjected to western blot to detect UAP56 protein and β-actin as control. Quantification of protein bands normalized to their loading control is shown at the bottom of the blots. (I) Purified RNA from total cell lysates in (G) were subjected to qPCR to measure viral mRNA levels. (J) Purified RNA from nuclear and cytoplasmic fractions from cells treated as in (G-J) were subjected to qPCR to measure viral mRNA levels in both fractions and determine their nuclear to cytoplasmic ratios (N/C). Control for cell fractionation is shown in S3 Fig. n=3. Graphs are mean +/−SD. *p<0.05, **p<0.01, ****p<0.0001.

FIG. 32 depicts compound 2 activity phenocopies down-regulation of the mRNA export factor UAP56. A549 cells or A549 cells stably expressing UAP56 E179A mutant were untreated or treated with control siRNA or with siRNA targeting the 3′UTR of UAP56 to knockdown endogenous UAP56 mRNA. Cells were then infected with WSN at MOI 2 for 8 h followed by RNA-FISH to detect poly(A) RNA (A-C) or smRNA-FISH to detect M (D- F), HA (G-I), and NS (J-L) mRNAs. For B-C (C, n=128 cells; UAP56-E197A+siRNA Control, n=117 cells; UAP56-E197A_siUAP56-3′UTR, n=170 cells). For E-F (C, n=121 cells; UAP56-E197A+siRNA Control, n=106 cells; UAP56-E197A siUAP56-3′UTR, n=108 cells). For H-I (C, n=124 cells; UAP56-E197A+siRNA Control, n=118 cells; UAP56-E197A siUAP56-3′UTR, n=151 cells). For K-L (C, n=113 cells; UAP56-E197A+siRNA Control, n=119 cells; UAP56-E197A_siUAP56-3′UTR, n=115 cells). Graphs show data points and mean +/- SD. *p<0.05, **p<0.01, ***p<0.001 ****p<0.0001.

FIG. 33 depicts compound 2 altering the levels and intracellular distribution of a subset of cellular mRNAs. Poly(A) RNA from total cell lysates, nuclear and cytoplasmic fractions untreated or treated with compound 2 was subjected to RNAseq analysis. Two biological duplicates were analyzed and the cut off is 1.5 fold for all analysis. RNAs selected were hits in both samples. (A) RNAs that are nuclear retained (yellow) or preferentially exported to the cytoplasm (light blue) are shown. Marked in red are RNAs whose total levels were not altered. Controls for fractionation are shown in S1 Table. (B) The number of RNAs that are up-regulated or down-regulated by compound 2 are shown. Marked in green are the number of RNAs known to be regulated by NS1 during infection. The identity of these RNAs are shown in S1 Table. (C-F) Selected mRNAs were also analyzed by qPCR to corroborate the RNAseq analysis. Relative mRNA levels and nuclear to cytoplasmic ratios of SPTLC3 (D), CEACAM19 (E), VTCN1 (F), and UQCC (G) were determined by qPCR from RNA obtained from total cell lysates, nuclear and cytoplasmic fractions treated with 0.1% DMSO or 2.5 μM compound 2 for 9 h. Three independent experiments were performed. C, control; Comp 2, Compound 2. Graphs show mean +/−SD. *p<0.05, **p<0.01, ***p<0.001 ****p<0.0001.

FIG. 34 depicts compound 2 inhibiting viral protein production and replication. (A) A549 cells were pre-treated with either 0.1% DMSO or 2.504 compound 2 before infection with A/WSN33 at MOI 2 for 8 h. Cell lysates were subjected to western blot analysis to detect viral proteins including PB1, PB2, PA, NA, NS1, M1, M2, and HA. β-Actin was used as a loading control. This blot is a representative of three independent experiments. (B-D) Effect of compound 2 on cell viability and viral replication of (B) A/WSN/33 (H1N1), (C) A/Vietnam/1203/04 (H5N1), and (D) A/Panama/99 (H3N2) influenza A virus strains. Cell viability was determined by the MTT assay in cells treated for 24 h (H1N1 and H5N1) or 48 h (H3N2). Viral titer was determined by plaque assay in cells infected for 24 h (H1N1 and H5N1) or 48 h (H3N2) at MOI 0.01. Three independent experiments were performed. Error bars are SD.

FIG. 35 depicts growth rate of NS1-BP knockout cells compared to wild-type cells. Cell growth of NS1-BP wild-type and knockout cells was monitored at 24, 48, 72, and 96 hours as determined by CellTiter-Glo. Four independent experiments were performed. Graph shows mean +/−SD. ***p<0.001, ****p<0.0001.

FIG. 36 depicts cluster analysis of confirmed hits. The 187 compounds identified for follow up studies are the most active members of 187 clusters. Within each active cluster, there are related analogs with lesser activity. The 187 clusters (arbitrarily numbered 1 to 187) are shown on the x-axis and the number of related analogs for each cluster plotted on the y-axis. Cluster size ranged from 1 to 32 members. Singleton clusters comprised 31% of the structural clusters (chemotypes). Compound 2 is a member of cluster 164 (indicated in red), which has 5 members. Clustering was performed with Pipeline Pilot v16 (Biovia, Inc.) using ECFP4 fingerprints.

FIG. 37 depicts control for the cell fractionation shown in FIG. 31. A549 cells were treated with 1 nM or 20 nM siRNA targeting the 3′UTR of the UAP56 mRNA or with control siRNA and then infected with WSN at MOI 2 for 8h. Purified RNA from total cell extract (A) or nuclear and cytoplasmic fractions (B) was subjected to qPCR to detect MALAT1 (a long non-coding RNA localized in the nucleus) as a nuclear marker. (C) Purified RNA from A was also used to detect total levels of 18S RNA or determine its nuclear to cytoplasmic distribution (D). 18S RNA is preferentially localized in the cytoplasm. Three independent experiments were performed. Graphs show mean +/−SD. Cyto, cytoplasm; Nuc, nucleus.

FIG. 38 depicts compound 2 inhibiting influenza virus replication in primary human bronchial epithelial cells at non-toxic concentrations. (A) Viral titer was determined by plaque assay in primary human bronchial epithelial cells (HBEC) infected with A/WSN/33 for 24 h in the absence or presence of compound 2 at the depicted concentrations. (B) Cell viability was monitored at 24 h after treatment with 0.1% DMSO or compound 2 at the depicted concentrations using CellTiter-Glo. Three independent experiments were performed. Graph shows mean +/−SD. **p<0.01. ***p<0.001, ****p<0.0001.

FIG. 39 depicts positive control for compound cytotoxicity. A549 cells were incubated with ivermectin, a compound present in our chemical library, at the depicted concentrations for 48 h. Cell viability was determined by the MTT assay. Three independent experiments were performed. Graph shows mean +/−SD. ***p<0.001.

FIG. 40 presents data showing that compound JMN3-003 (N-aryl mercaptobenzimidazole) does not inhibit viral mRNA nuclear export. (A) Structure of compound JMN3-003. (B) smRNA-FISH followed by fluorescence microscopy was performed to detect M mRNA in cells treated with 0.1% DMSO or 2.5 μM JMN3-003. These treatments started 1 hour before infection with WSN at MOI 2 for 8 h. Total fluorescence intensity (C) or nuclear to cytoplasmic fluorescence intensity (N/C ratio) (D) of M mRNA was quantified for images in B. For both C and D (C, n=123 cells; JMN3-003, n=141 cells). Graphs show data points and mean +/−SD. ****p<0.0001. This compound decreased total viral M mRNA levels but did not retain viral M mRNA in the nucleus as compound 2.

### DETAILED DESCRIPTION

It will be appreciated that numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein.

Section headings as used in this section and the entire disclosure herein are merely for organizational purposes and are not intended to be limiting.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. As used herein, the following terms have the meanings ascribed to them unless specified otherwise.

Articles “a” and “an” are used herein to refer to one or to more than one (i.e., at least one) of the grammatical object of the article. By way of example, “an element” means at least one element and can include more than one element.

“About” is used herein to provide flexibility to a numerical range endpoint by providing that a given value may be “slightly above” or “slightly below” the endpoint without affecting the desired result.

As used herein, the term “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, the term “inhibit” or “inhibiting” refers to reduction in the amount, levels, density, turnover, association, dissociation, activity, signaling, or any other feature associated with the protein.

As used herein, “administration” of a disclosed compound encompasses the delivery to a subject of a compound as described herein, or a prodrug or other pharmaceutically acceptable derivative thereof, using any suitable formulation or route of administration, as discussed herein.

The term “effective amount” or “therapeutically effective amount” refers to an amount sufficient to effect beneficial or desirable biological and/or clinical results including, but not limited to, disease treatment, as illustrated below. In some embodiments, the amount is that effective for detectable reduction of pain. In some embodiments, the amount is that effective for alleviating, reducing or eliminating a pathologic pain.

The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The term also applies to a dose that will induce a particular response in target cells, e.g., reduction of cell migration. The specific dose will vary depending on, e.g., the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). In some embodiments, the subject comprises a subject suffering from an influenza viral infection.

The present disclosure illustrates that gene knockout of the cellular protein NS1-BP, a constituent of the M mRNA speckle-export pathway and a binding partner of the virulence factor NS1 protein, inhibits M mRNA nuclear export without altering bulk cellular mRNA export, thus providing an avenue to preferentially target influenza virus. Through a high-content, image-based chemical screen, inhibitors of viral mRNA biogenesis and nuclear export were identified that exhibited no significant activity towards bulk cellular mRNA at non-cytotoxic concentrations. Among the hits is a small molecule that preferentially inhibits nuclear export of a subset of viral and cellular mRNAs without altering bulk cellular mRNA export. These findings underscore specific nuclear export requirements for viral mRNAs and phenocopy down-regulation of the mRNA export factor UAP56. This RNA export inhibitor impaired replication of diverse influenza A virus strains at non-toxic concentrations. Thus, this screening strategy yielded compounds that alone or in combination may serve as leads to new ways of treating influenza virus infection and are novel tools for studying viral RNA trafficking in the nucleus.

The present disclosure provides methods and compositions for the treatment or prevention of influenza. In particular, the present disclosure provides compounds that prevent influenza virus replication through inhibition of influenza viral mRNA nuclear export, as well as methods for the treatment or prevention of influenza that include administration of such compounds.

According to one aspect of the present disclosure, there is provided a method of treating an influenza viral infection in a subject in need thereof. Such method comprises administering to the subject a therapeutically effective amount of an inhibitor of influenza viral M mRNA nuclear export.

In one embodiment, the inhibitor targets a cellular protein in viral M mRNA speckle-export pathway. By way of non-limiting example, the cellular protein is a binding partner of viral NS1 protein. By way of non-limiting example, the cellular protein targeted by the inhibitor is NS1-BP.

Since Influenza A viruses are human pathogens with limited therapeutic options, it is crucial to devise strategies for the identification of new classes of antiviral medications. The Influenza A virus genome is constituted of 8 RNA segments. Two of these viral RNAs are transcribed into mRNAs that are alternatively spliced. The M1 mRNA encodes the M1 protein but is also alternatively spliced to yield the M2 mRNA during infection. M1 to M2 mRNA splicing occurs at nuclear speckles, and M1 and M2 mRNAs are exported to the cytoplasm for translation. M1 and M2 proteins are critical for viral trafficking, assembly, and budding. Nuclear speckles are known to be storage sites for splicing and other RNA processing factors, and this process requires key viral-host interactions for both splicing and nuclear export of the viral M2 mRNA. This suggests a pathway in which the viral NS1 protein interacts with the cellular NS1-BP protein, which in turn binds hnRNP K to target the M1 mRNA from the nucleoplasm to nuclear speckles. At this nuclear body, the U1 snRNP and/or dissociation of NS1 induces a remodeling of the protein-RNA complex in a manner that hnRNP K recruits U1 snRNP to the M2 5′ splice site on M1 mRNA to mediate splicing. Then, NS1 and NS1-BP together with key members of the mRNA nuclear export machinery (the RNA helicase UAP56 and the mRNA export factor Aly/REF) promote nuclear export of M1 and M2 mRNAs.

Since this splicing-export pathway through nuclear speckles does not impact bulk mRNA but only a subset of viral and cellular mRNAs, chemical compounds antagonizing this process would have the potential of not being overly toxic and could inhibit virus replication. Through an image-based chemical screen using single-molecule RNA-FISH to detect the viral M mRNA (M1 and M2 mRNAs), chemical compounds were identified that would inhibit different steps of this speckle-export intranuclear pathway yet would not significantly compromise bulk poly(A) RNA.

Accordingly, in one embodiment, the inhibitor is a compound comprising a structural formula selected from the group consisting of Structural Formula I, Structural Formula II, Structural Formula III, Structural Formula IV, Structural Formula V, Structural Formula VI, Structural Formula VII, Structural Formula VIII, Structural Formula IX, Structural Formula X, Structural Formula XI, Structural Formula XII, Structural Formula XIII, Structural Formula XIV, Structural Formula XV, Structural Formula XVI, Structural Formula XVII, Structural Formula XVIII, Structural Formula XIX, Structural Formula XX, Structural Formula XXI, Structural Formula XXII, Structural Formula XXIII, Structural Formula XIV, Structural Formula XV, Structural Formula XVI, Structural Formula XVII, Structural Formula XVIII, and Structural Formula XXIX, or a pharmaceutically acceptable salt, solvate, hydrate, prodrug, or derivative thereof.

In one embodiment, the inhibitor is a compound comprising Structural Formula I below:

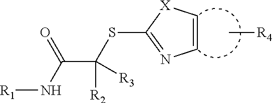

or a pharmaceutically acceptable salt, solvate, hydrate, prodrug, or derivative thereof; wherein R₁ is an unsubstituted or substituted aryl or heteroaryl; R₂ and R₃ are either the same or different and are selected from H or alkyl; X is selected from NH, NR₅, O, and S; R₄ is appended to an optional ring as part of a benzo-fused heteroaryl and is selected from H, alkyl or halogen; and R5 is an alkyl or aryl.

In one embodiment, the inhibitor is a compound comprising Structural Formula II below:

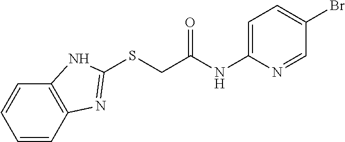 [SMILES: O=C(CSc1nc2ccccc2[nH]1)Nc1ccc(Br)cn1]

or a pharmaceutically acceptable salt, solvate, hydrate, prodrug, or derivative thereof.

By way of non-limiting example, the inhibitor is BrC1=CC═C(NC(═O)CSC2=NC3=C(N2)C═CC=C3)N=C1

In one embodiment, the inhibitor is a compound comprising Structural Formula III below:

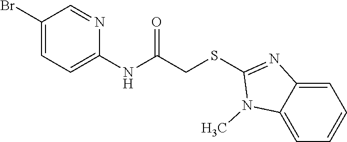 [SMILES: Cn1c(SCC(=O)Nc2ccc(Br)cn2)nc2ccccc21]

or a pharmaceutically acceptable salt, solvate, hydrate, prodrug, or derivative thereof.

By way of non-limiting example, the inhibitor is CN1C(SCC(═O )NC2=CC═C(Br)C=N2)=NC2=CC═CC=C12.

In one embodiment, the inhibitor is a compound comprising Structural Formula IV below:

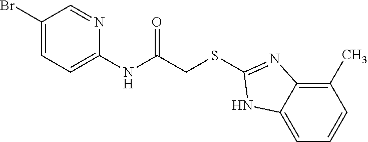 [SMILES: Cc1cccc2[nH]c(SCC(=O)Nc3ccc(Br)cn3)nc12]

or a pharmaceutically acceptable salt, solvate, hydrate, prodrug, or derivative thereof.

By way of non-limiting example, the inhibitor is CC1=C2N═C(NC2=CC=C1)SCC(═O)NC1=CC═C(Br)C=N1.

The method disclosed above and herein can be used to treat an influenza viral infection caused by different influenza viruses. In one embodiment, the influenza viral infection may be caused by an influenza A virus (IAV). By way of non-limiting example, the influenza virus A is subtype H1N1, H2N2, H3N2, or H5N1. In one embodiment, the influenza viral infection may be caused by an influenza B virus (IBV).

In one embodiment, at least one additional therapeutic agent may be further administered to the subject in need thereof. By way of non-limiting example, the additional therapeutic agent may be Rapivab, Relenza, Tamiflu, Xofluza, or a combination thereof.

When used to treat or prevent such diseases (e.g., flu), the compounds described herein may be administered singly, as mixtures of one or more compounds or in mixture or combination with other agents useful for treating such diseases and/or the symptoms associated with such diseases. In one embodiment, a compound comprising the Structural Formula II may be administered together with a compound comprising Structural Formula III and/or a compound comprising Structural Formula IV. The compounds may also be administered in mixture or in combination with agents useful to treat other disorders or maladies. The compounds may be administered in the form of compounds per se, or as pharmaceutical compositions comprising a compound.

In one embodiment, the compounds may be administered in combination with a therapeutic treatment modality. By way of non-limiting example, the therapeutic treatment modality may be a flu vaccine.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically. The exact nature of the carrier, diluent, excipient or auxiliary will depend upon the desired use for the composition, and may range from being suitable or acceptable for veterinary uses to being suitable or acceptable for human use. The composition may optionally include one or more additional compounds.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

In one embodiment, the compounds described above and herein may be administered orally, buccally, sublingually, rectally, intravenously, intramuscularly, topically, auricularly, conjunctivally, nasally, via inhalation, or subcutaneously.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC50 of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

Another aspect of the present disclosure provides a method of preventing an influenza viral infection in a subject in need thereof. Such method comprises administering to the subject a therapeutically effective amount of an inhibitor of influenza viral M mRNA nuclear export. In one embodiment, the inhibitor targets a cellular protein in viral M mRNA speckle-export pathway. By way of non-limiting example, the cellular protein is a binding partner of viral NS1 protein. By way of non-limiting example, the cellular protein is NS1-BP.

In one embodiment, the inhibitor is a compound comprising a structural formula selected from the group consisting of Structural Formula I, Structural Formula II, Structural Formula III, Structural Formula IV, Structural Formula V, Structural Formula VI, Structural Formula VII, Structural Formula VIII, Structural Formula IX, Structural Formula X, Structural Formula XI, Structural Formula XII, Structural Formula XIII, Structural Formula XIV, Structural Formula XV, Structural Formula XVI, Structural Formula XVII, Structural Formula XVIII, Structural Formula XIX, Structural Formula XX, Structural Formula XXI, Structural Formula XXII, Structural Formula XXIII, Structural Formula XIV, Structural Formula XV, Structural Formula XVI, Structural Formula XVII, Structural Formula XVIII, and Structural Formula XXIX, or a pharmaceutically acceptable salt, solvate, hydrate, prodrug, or derivative thereof.

In one embodiment, the inhibitor is a compound comprising Structural Formula I below:

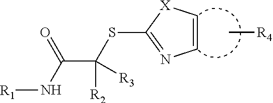

or a pharmaceutically acceptable salt, solvate, hydrate, prodrug, or derivative thereof; wherein R₁ is an unsubstituted or substituted aryl or heteroaryl; R₂ and R₃ are either the same or different and are selected from H or alkyl; X is selected from NH, NR₅, O, and S; R₄ is appended to an optional ring as part of a benzo-fused heteroaryl and is selected from H, alkyl or halogen; and R5 is an alkyl or aryl.

In one embodiment, the inhibitor is a compound comprising Structural Formula II below:

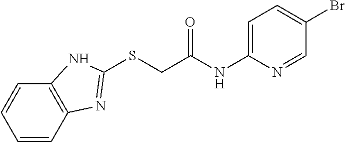 [SMILES: O=C(CSc1nc2ccccc2[nH]1)Nc1ccc(Br)cn1]

or a pharmaceutically acceptable salt, solvate, hydrate, prodrug, or derivative thereof.

By way of non-limiting example, the inhibitor is BrC1=CC═C(NC(═O)CSC2=NC3=C(N2)C═CC=C3)N=C1.

In one embodiment, the inhibitor is a compound comprising Structural Formula III below:

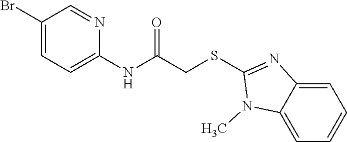 [SMILES: Cn1c(SCC(=O)Nc2ccc(Br)cn2)nc2ccccc21]

or a pharmaceutically acceptable salt, solvate, hydrate, prodrug, or derivative thereof.

By way of non-limiting example, the inhibitor is CN1C(SCC(═)NC2=CC═C(Br)C=N2)=NC2=CC═CC=C12.

In one embodiment, the inhibitor is a compound comprising Structural Formula IV below:

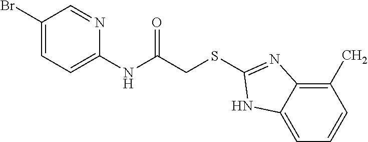 [SMILES: Cc1cccc2[nH]c(SCC(=O)Nc3ccc(Br)cn3)nc12]

or a pharmaceutically acceptable salt, solvate, hydrate, prodrug, or derivative thereof.

By way of non-limiting example, the inhibitor is CC1=C2N═C(NC2=CC=C1)SCC(═O)NC1=CC═C(Br)C=N1.

The method disclosed above and herein can be used to prevent an influenza viral infection caused by different influenza viruses. In one embodiment, the influenza viral infection may be caused by an influenza A virus (IAV). By way of non-limiting example, the influenza virus A is subtype H1N1, H2N2, H3N2, or H5N1. In one embodiment, the influenza viral infection may be caused by an influenza B virus (IBV).

In one embodiment, at least one additional therapeutic agent may be further administered to the subject in need thereof. By way of non-limiting example, the additional therapeutic agent may be Rapivab, Relenza, Tamiflu, Xofluza, or a combination thereof.

When used to treat or prevent such diseases (e.g., flu), the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies. The compounds may be administered in the form of compounds per se, or as pharmaceutical compositions comprising a compound.

In one embodiment, the compounds may be administered in combination with a therapeutic treatment modality. By way of non-limiting example, the therapeutic treatment modality may be a flu vaccine.

In one embodiment, the inhibitor or at least one additional therapeutic agent is administered orally, buccally, sublingually, rectally, intravenously, intramuscularly, topically, auricularly, conjunctivally, nasally, via inhalation, or subcutaneously.

According to still another aspect of the present disclosure, there is provided a kit for treating or preventing an influenza viral infection. Such kit comprises a therapeutically effective amount of an inhibitor of influenza viral M mRNA nuclear export, a means of administering the inhibitor, and instructions for use.

In one embodiment, the inhibitor is a compound comprising a structural formula selected from the group consisting of Structural Formula I, Structural Formula II, Structural Formula III, Structural Formula IV, Structural Formula V, Structural Formula VI, Structural Formula VII, Structural Formula VIII, Structural Formula IX, Structural Formula X, Structural Formula XI, Structural Formula XII, Structural Formula XIII, Structural Formula XIV, Structural Formula XV, Structural Formula XVI, Structural Formula XVII, Structural Formula XVIII, Structural Formula XIX, Structural Formula XX, Structural Formula XXI, Structural Formula XXII, Structural Formula XXIII, Structural Formula XIV, Structural Formula XV, Structural Formula XVI, Structural Formula XVII, Structural Formula XVIII, and Structural Formula XXIX, or a pharmaceutically acceptable salt, solvate, hydrate, prodrug, or derivative thereof.

In one embodiment, the inhibitor is a compound comprising Structural Formula I below:

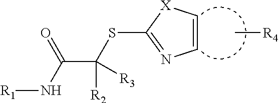

or a pharmaceutically acceptable salt, solvate, hydrate, prodrug, or derivative thereof; wherein R₁ is an unsubstituted or substituted aryl or heteroaryl; R₂ and R₃ are either the same or different and are selected from H or alkyl; X is selected from NH, NR₅, O, and S; R₄ is appended to an optional ring as part of a benzo-fused heteroaryl and is selected from H, alkyl or halogen; and R5 is an alkyl or aryl.

In one embodiment, the inhibitor is a compound comprising Structural Formula II below:

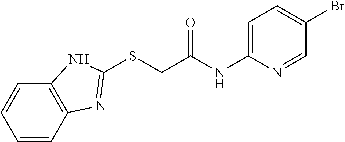 [SMILES: O=C(CSc1nc2ccccc2[nH]1)Nc1ccc(Br)cn1]

or a pharmaceutically acceptable salt, solvate, hydrate, prodrug, or derivative thereof.

By way of non-limiting example, the inhibitor is BrC1=CC═C(NC(═O)CSC2=NC3=C(N2)C═CC=C3)N=C1.

In one embodiment, the inhibitor is a compound comprising Structural Formula III below:

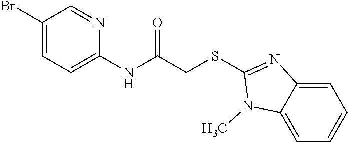 [SMILES: Cn1c(SCC(=O)Nc2ccc(Br)cn2)nc2ccccc21]

or a pharmaceutically acceptable salt, solvate, hydrate, prodrug, or derivative thereof.

By way of non-limiting example, the inhibitor is CN1C(SCC(═O)NC2=CC═C(Br)C=N2)=NC2=CC═CC=C12.

In one embodiment, the inhibitor is a compound comprising Structural Formula IV below:

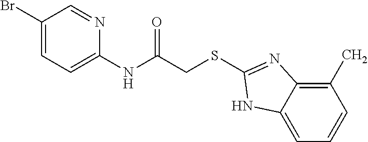 [SMILES: Cc1cccc2[nH]c(SCC(=O)Nc3ccc(Br)cn3)nc12]

or a pharmaceutically acceptable salt, solvate, hydrate, prodrug, or derivative thereof.

By way of non-limiting example, the inhibitor is CC1=C2N═C(NC2=CC=C1)SCC(═O)NC1=CC═C(Br)C=N1.

In one embodiment, the kit further comprises at least one additional therapeutic agent. By way of non-limiting example, the additional therapeutic agent may be Rapivab, Relenza, Tamiflu, Xofluza, or a combination thereof.

In some embodiments, the kit is packaged in a container with a label affixed to the container or included in the package that describes use of the compounds described herein. Exemplary containers include, but are not limited to, a vessel, vial, tube, ampoule, bottle, flask, and the like. It is contemplated that the container is made from material well-known in the art, including, but not limited to, glass, polypropylene, polystyrene, and other plastics. In various aspects, the compounds are packaged in a unit dosage form. In various aspects, the kit contains a label and/or instructions that describes use of the contents for pain treatment.

The following Examples are provided by way of illustration and not by way of limitation.

### EXAMPLES

### Materials and Methods

### Cell Culture

Human lung adenocarcinoma epithelial cells (A549) and MDCK cells, obtained from ATCC (American Type Culture Collection), were maintained in high-glucose DMEM (Gibco), 10% FBS (Sigma), and 100 units/mL Pen/Strep antibiotics at 37° C. with 5% CO2. Primary human bronchial epithelial cells were cultured as previously reported (Peters-Hall, et al., 2019, The FASEB Journal, 00: 1-13). A549 cells stably expressing UAP56 E179A mutant were generated according to Hondele et al. (2019, Nature 573(7772): 144-8).

Transfections and siRNAs

siRNAs were reverse transfected with A549 cells using RNAiMAX (Invitrogen) in OptiMEM (ThermoFisher) by the manufacturer's instructions. After 24 h transfection, media was replaced with growth media. Knockdown was allowed to continue for 48 h before compound treatment or infections occurred. siRNAs used include UAP56 and MISSION siRNA Universal Negative Control #2 (Sigma-Aldrich), ON-TARGETplus siRNAs against SON and ON-TARGETplus Non-targeting Control #2 (Dharmacon, ThermoFisher), and 3′UTR siUAP56 sequence 5′-GCUUCCAUCUUUUGCAUCAUU-3′ (SEQ ID NO: 73) (Dharmacon).

### NS1-BP Knockout Cell Line

The NS1-BP gene was knocked out in A549 cells by genome editing using CRISPR-Cas9. In brief, the genomic target oligos (Forward: CACCGTGCTTATGGCCATTCTCACG (SEQ ID NO: 74), Reverse: AAACCGTGAGAATGGCCATAAGCAC (SEQ ID NO: 75)) were cloned into a lentiCRISPRv2 vector. The plasmid was co-transfected into HEK293T cells, obtained from ATCC (American Type Culture Collection), with the packaging plasmids pVSVg and psPAX2, generating lentivirus to infect A549 cells. Then, cells were clonally selected using puromycin (1.0 μg/ml) for 7 days followed by 3 days without selection for expansion. Clones were isolated and expanded to generate lysates for western blot analysis using anti-NS1-BP antibody. Candidate clones were subjected to genomic sequencing using amplicons flanking the sgRNA-target site. Growth rates were determined by measuring ATP levels. Cells were tested at 24 h, 48 h, 72 h, and 96 h after plating equal number of NS1-BP^{+/+} and NS1-BP^{31/-} cells. ATP was measured by luminescence using CellTiter-Glo (Promega) according to the manufacturer's instructions.

### Viruses

Influenza A viruses (A/WSN/33, A/Vietnam/1203/04, A/Panama/99) were generated in embryonated eggs or in MDCK cells after growth from a clonal population of virus at low multiplicity of infection to avoid accumulation of defective virus particles. In MDCK cells, virus was amplified at MOI 0.1-0.001 in infection media containing EMEM (ATCC, 30-2003), 10 mM HEPES (Gibco), 0.125% BSA (Gibco), 0.5 μg/mL TPCK trypsin (Worthington Biomedical Corporation). Cells were incubated with virus for 1 hour at 37° C., then washed before amplification in infection media. After cell death was observed at 48-72 hours post-infection, supernatants were centrifuged at 1,000×g for 10 minutes to remove cell debris, aliquoted, and stored at −80° C. All virus stocks are controlled for an appropriate ratio of HA/PFU titer and sequenced by RNAseq to confirm the full sequence of the virus.

### Viral Replication and Cytotoxicity Assays

A549 cells were infected with A/WSN/33 and A/Vietnam/1203/04 at MOI 0.01, or with A/Panama/99 at MOI 0.1 in the absence or presence of compound 2 at concentrations depicted in the figures. Supernatants were collected from each condition 24 h post-infection and viral particles were tittered by plaque assay as following: MDCK cells were seeded in 6-well plates to reach confluency the next day. At confluency, ten-fold serial dilutions of each sample's supernatant were diluted in PBS containing 100 units/mL Pen/Strep antibiotics, 0.2% BSA, 0.9 mM CaCl2, and 1.05 mM MgCl2. After infection with each dilution, cells were overlaid with a 1:1 mixture of 2×-15 media and 2% Oxiod Agar (Final concentration of 1×L-15 media and 1% Agar). Plaques formed at 24 h for A/WSN/33 and A/Vietnam/1203/04, or 48 h for A/Panama/99 were counted and titers determined. Primary human bronchial epithelial cells were infected with A/WSN/33 at MOI 0.1 for 24 h in the absence or presence of compound 2 at depicted concentrations. Supernatants were subjected to plaque assays as described above. Cytotoxicity was also performed using the MTT assay (Roche), according to the manufacturer's instructions, concurrent with viral replication assay.

smRNA-FISH

smRNA-FISH was performed as previously described (Mor et al., 2016, Nat Microbiol. 1(7): 16069), which includes the sequences of M1 and NS1 probes except for the HA probes that are listed in Table 1 below. Briefly, cells were grown on glass coverslips (Fisherbrand, FisherScientific) coated with 1mL of 0.1% gelatin (Sigma-Aldrich). Cells were fixed with 4% paraformaldehyde (PFA, Electron Microscopy Sciences) in PBS for 15 min before incubation in 70% ethanol for 12 h at 4° C. Coverslips were then placed in wash buffer for 5 min, containing Nuclease Free Water, 2×SSC Buffer (Sigma), and 10% formamide (Sigma). The coverslips where then removed and incubated in hybridization buffer containing FISH probe. Hybridization occurred at 37° C. for 4 h, then cells were washed with wash buffer for 30 min at 37° C. Coverslips were then washed twice for 5 min in PBS and stained with 1 μg/ml Hoechst 33258 (Molecular Probes/Life Technologies) for 10 min. Coverslips were briefly washed with PBS before mounting in ProLong Gold antifade reagent (Life Technologies).

Table 1 lists the forty-eight, 20 nt DNA probes labeled with Quasar 570 (BIOSEARCH TECHNOLOGIES) that were designed to hybridize with the Influenza WSN full length HA mRNA.

TABLE 1

| Probe # | Probe (5′ → 3′) |
|---|---|
| 1 (SEQ ID NO: 1) | catattgtgtctgcatctgt |
| 2 (SEQ ID NO: 2) | ttgagttgttcgcatggtag |
| 3 (SEQ ID NO: 3) | gccacattcttctcgaatat |
| 4 (SEQ ID NO: 4) | gtcttcgagcaggttaacag |
| 5 (SEQ ID NO: 5) | ttacatagtttcccgttgtg |
| 6 (SEQ ID NO: 6) | caattgtagtggggctattc |
| 7 (SEQ ID NO: 7) | catccggtgatgttacattt |
| 8 (SEQ ID NO: 8) | tgagtcgcattctggatttc |
| 9 (SEQ ID NO: 9) | cattctcagagtttggtgtt |
| 10 (SEQ ID NO: 10) | tcagttcctcatagtcgatg |
| 11 (SEQ ID NO: 11) | gatactgagctcaattgctc |
| 12 (SEQ ID NO: 12) | ccatgaactttccttgggaa |
| 13 (SEQ ID NO: 13) | gagcatgatactgttactcc |
| 14 (SEQ ID NO: 14) | gtaaaaactgctttttcccc |
| 15 (SEQ ID NO: 15) | ttcgtcagccatagcaaatt |
| 16 (SEQ ID NO: 16) | aattggtcagctttgggtat |
| 17 (SEQ ID NO: 17) | tttccctttattgttcacat |
| 18 (SEQ ID NO: 18) | tgatgaacaccccatagtac |
| 19 (SEQ ID NO: 19) | gggtgaatctcctgttataa |
| 20 (SEQ ID NO: 20) | cccatgttgatcttttactt |
| 21 (SEQ ID NO: 21) | gcaaggtccagtaatagttc |
| 22 (SEQ ID NO: 22) | tattagattaccagttgcct |
| 23 (SEQ ID NO: 23) | tcagtgcgaaagcataccat |
| 24 (SEQ ID NO: 24) | tgatgatgccggactcaaac |
| 25 (SEQ ID NO: 25) | tcatgcattgacgcgtttga |
| 26 (SEQ ID NO: 26) | gtgtttgacacttcgtgtta |
| 27 (SEQ ID NO: 27) | gattgctgtttatagatccc |
| 28 (SEQ ID NO: 28) | gactgggtgtatattctgga |
| 29 (SEQ ID NO: 29) | tgacatattttgggcactct |
| 30 (SEQ ID NO: 30) | gtaaccatcctcaatttggt |
| 31 (SEQ ID NO: 31) | ggatgggatgtttcttagtc |
| 32 (SEQ ID NO: 32) | ctccaaatagacctctgtat |
| 33 (SEQ ID NO: 33) | cccctcaataaaaccagcaa |
| 34 (SEQ ID NO: 34) | aaccataccatccatctatc |
| 35 (SEQ ID NO: 35) | ttttttgatccgctgcatag |
| 36 (SEQ ID NO: 36) | tttgtaatcccgttaatggc |
| 37 (SEQ ID NO: 37) | ctcgataacagagttcacct |
| 38 (SEQ ID NO: 38) | tgtccaaatgtccagaaacc |
| 39 (SEQ ID NO: 39) | ggctttttactttctcgtac |
| 40 (SEQ ID NO: 40) | tccgatttctttggcattat |
| 41 (SEQ ID NO: 41) | tcattgtcacacttgtggta |
| 42 (SEQ ID NO: 42) | aagtcccatttcttacactt |
| 43 (SEQ ID NO: 43) | ctatcttttccctgttcaac |
| 44 (SEQ ID NO: 44) | cccattgattccaatttcac |
| 45 (SEQ ID NO: 45) | tggcgacagttgagtagatc |
| 46 (SEQ ID NO: 46) | gagaccaaaagcaccagtga |
| 47 (SEQ ID NO: 47) | acatccagaaactgattgcc |
| 48 (SEQ ID NO: 48) | atgcatattctgcactgcaa |

### High-Throughput Screen and Statistics

To identify chemical inhibitors of viral M mRNA processing and nuclear export, A549 cells were treated with 232,500 chemical compounds available from the University of Texas Southwestern Medical Center High Throughput Screening core facility. Cells were treated with 2.5 μM compound for 30 minutes and incubated at 37° C. in 5% CO2. Cells were then infected with influenza A/WSN/33 virus at MOI of 2 and returned to incubation as before. At 7.5 hours post-infection, cells were fixed with 4% paraformaldehyde and subjected to RNA-FISH. To localize M mRNA, forty-five FISH probes labeled with Quasar 570 were used that cover the entire M mRNA segment, as previously reported (Mor et al., 2016, Nat Microbiol. 1(7): 16069). Nuclei were stained with 1 μg/m1 Hoechst 33342 dye. M mRNA distribution between the nucleus and the cytoplasm was detected using the IN Cell Analyzer 6000 (GE Healthcare, Marlborough MA). Multiple fields per well were taken at 20× magnification using the Hoechst and dsRed widefield fluorescence filters. Image analysis was performed in a GE IN Cell Analyzer Workstation 3.7.3 (GE Healthcare) using the multi-target analysis template. Individual nuclei were segmented using a top-hat filter on the Hoechst channel with the default sensitivity setting. For samples detecting the M1 mRNA, the cell body was segmented using the region growing method on the M1 mRNA channel. This method uses the nuclei as the seed and then expands outwards until the edge of the stain is reached. For samples detecting poly(A) RNA, the poly(A) RNA channel was instead used to define the cell body region using the region growing method. For each segmented nucleus and cell pair, the mean and total signal intensities of the nuclear and cytoplasmic chambers were calculated for the poly(A) RNA (where applicable) and M1 mRNA channels. The mean nuclear to mean cytoplasmic (N/C) ratio was then calculated for both mRNA probes for each cell. Finally, the average N/C ratios per well were calculated and used for hit identification. The results were imported into the GeneData Screener™ (Basel, Switzerland; version 13.0.6) software analysis suite to normalize and summarize the overall M mRNA intensity as well as nuclear to cytosolic ratio in terms of a Z-score as previously described (Zhang et al., 2015, Science 348: 6240; Wu et al., 2008, Journal of Biomolecular Screening, 13(2): 159-67).

In the primary screen, compounds with a robust Z-score of less than −3 for intensity were considered hits affecting virus replication. Compounds with a Z-score greater than 3 in the nuclear/cytosolic ratio were selected as hits for inhibition of nuclear export. Any compound that lowered the nuclear count to a Z-score of −3 or lower was considered cytotoxic and not included in follow-up experiments. Compounds (1,125) that had the highest activity were selected for confirmation and retested in triplicate at a compound concentration of 2.5 μM. All imaging confirmation and follow-up assays included a bulk poly(A) RNA probe linked to Quasar 670 for FISH imaging. As with the M mRNA probe, total intensity and N/C ratio were also measured for the poly(A) RNA probe. The 600 compounds with the highest activity from the confirmation assay were subjected to 12-point dose response curves ranging from 0.5 nM to 50 μM at 0.5 log dose intervals. Of the 600 compounds tested, 413 compounds had a measureable effect on bulk poly(A) RNA and were excluded from further testing. The remaining 187 compounds that inhibited viral mRNA nuclear export and/or decreased viral mRNA levels but had no substantial effect on the host cell poly(A) RNA were categorized into 3 major phenotypes. These include 22 compounds that retained viral M mRNA in the nucleus, 33 compounds that decreased viral M mRNA levels, and 132 compounds that decreased overall levels and inhibited nuclear export of viral M mRNA. Clustering analysis of confirmed hits was performed with Pipeline Pilot v16 (Biovia, Inc.) using ECFP4 fingerprints (Rogers and Hahn, 2010, J Chem Inf Model, 50(5):742-54).

### Image Quantification and Statistics

Total cell fluorescence intensity or fluorescence intensity in the nucleus and cytoplasm analysis was conducted. Images deconvolved with AutoQuant software were analyzed using Imaris (Bitplane). The Surfaces tool was used to segment fluorescence within the cytoplasm and nucleus of each cell quantified. Statistical analyses for imaging studies and qPCR data in the figures mentioned above were performed using the two-sample, two-tailed, t-test.

### Compounds

Compound 2-thiobenzimidazole was initially purchased from TimTec (HTS04595) as well as synthesized in-house. Comparative compound JMN3-003 was synthesized as previously described (Moore et al., 2013, J Org. Chem. 9: 197-203). All compounds were dissolved in dimethylsulfoxide (DMSO). Compound 2 was synthesized and characterized as following:

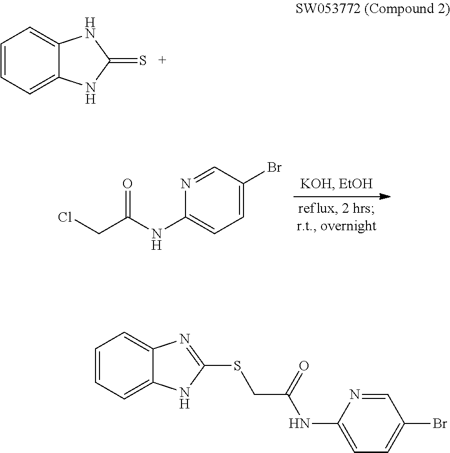

2-((1H-benzo[d]imidazol-2-yl)thio)-N-(5-bromopyridin-2-yl)acetamide

A mixture of 2-mercaptobenzimidazole (30.0 mg, 0.2 mmol, 1.0 equiv.) and crushed potassium hydroxide (11.2 mg, 0.2 mmol, 1.0 equiv.) in 2 ml of ethanol was kept at reflux for 2 hours. The reaction mixture was cooled down to room temperature, N-(5-bromopyridin-2-yl)-2-chloroacetamide (49.9 mg, 0.2 mmol, 1.0 equiv.) was added, and the reaction was stirred for overnight. The resulting reaction mixture was concentrated under reduced pressure. 2.0 ml of saturated ammonium chloride solution and 2.0 ml of dichloromethane were added to the residue. The organic layer was separated, washed with 2.0 ml of brine, then dried over sodium sulfate, filtered and concentrated under reduced pressure. The crude was further purified by silica gel chromatography using 60% of ethyl acetate in hexane to afford 54 mg white solid as product, yield 74%.

### ¹H NMR (CDCl3, 400 MHz)

δppm 8.31-8.24 (m, 1H), 8.03 (d, J=8.8 Hz, 1H), 7.72 (ddd, J=8.9, 2.8, 1.5 Hz, 1H), 7.48 (br, 2H), 7.20-7.08 (m, 3H), 4.03 (s, 2H).

### ¹³C NMR (CDCl3, 400 MHz)

δppm 168.23, 149.92, 149.48, 148.78, 140.57, 122.95, 122.35, 115.53, 114.86, 109.97, 36.24.

### MS

MS (ESI) m/z=363.0 ([M+H]⁺), C₁₄H₁₁BrN₄OS requires 363.0.

### Measurement of Cellular ATP Levels

ATP was measured by luminescence using the CellTiter-Glo kit (Promega) according to the manufacturer's instructions.

### RNA Purification and RT-qPCR

Total RNA was isolated from A549 cells using the RNeasy Plus Mini Kit (Qiagen) and reverse transcribed into cDNA by SuperScript II reverse transcriptase (Invitrogen), each according to the manufacturers' protocols. Samples were then amplified in a LightCycler 480 quantitative real-time PCR (qPCR) system (Roche) using SYBR Green I (Roche) and sequence specific primers. RT-PCR Primer Sequences are listed below:

| | |
|---|---|
| M1 | Forward- ATCAGACATGAGAACAGAATGG (SEQ ID NO: 49) |
| | Reverse- TGCCTGGCCTGACTAGCAATATC (SEQ ID NO: 50) |
| M2 | Forward: CGAGGTCGAAACGCCTATCAGAAAC (SEQ ID NO: 51) |
| | Reverse: CCAATGATATTTGCTGCAATGACGAG (SEQ ID NO: 52) |
| NS1 | Forward: TGGAAAGCAAATAGTGGAGCG (SEQ ID NO: 53) |
| | Reverse: GTAGCGCGATGCAGGTACAGAG (SEQ ID NO: 54) |
| NS2 | Forward: CAAGCTTTCAGGACATACTGATGAG (SEQ ID NO: 55) |
| | Reverse: CTTCTCCAAGCGAATCTCTGTAGA (SEQ ID NO: 56) |
| HA | Forward: TCTATTTGGAGCCATTGCTGG (SEQ ID NO: 57) |
| | Reverse: TGCTTTTTTGATCCGCTGCA (SEQ ID NO: 58) |
| 18S | Forward: GTAACCCGTTGAACCCCATT (SEQ ID NO: 59) |
| | Reverse: CCATCCAATCGGTAGTAGCG (SEQ ID NO: 60) |
| β-actin | Forward: CCGCGAGAAGATGACCCAGAT (SEQ ID NO: 61) |
| | Reverse: CGTTGGCACAGCCTGGATAGCAACG (SEQ ID NO: 62) |
| SPTLC3 | Forward: GGAATTGGAACCCTGTTTGGC (SEQ ID NO: 63) |
| | Reverse: GTCTCTGATTCGCATGTAAAGGT (SEQ ID NO: 64) |
| CEACAM19 | Forward: GCCCAGCCTACAGACAGTG (SEQ ID NO: 65) |
| | Reverse: GCAGCAAGAGATCCAATGATGG (SEQ ID NO: 66) |
| VTCN1 | Forward: TCTGGGCATCCCAAGTTGAC (SEQ ID NO: 67) |
| | Reverse: TCCGCCTTTTGATCTCCGATT (SEQ ID NO: 68) |
| UQCC | Forward: GGAGAAAACTGACTTCGAGGAAT (SEQ ID NO: 69) |
| | Reverse: TCCAGACGTGGAGTAGGGTTA (SEQ ID NO: 70) |
| UAP56 | Forward: CTTTGAGCATCCGTCAGAAGT (SEQ ID NO: 71) |
| | Reverse: AGTGTGACACATCACCAGTACA (SEQ ID NO: 72) |

### Cell Fractionation and RNAseq Analysis

Cells were treated with 0.1% DMSO or 2.5 μM compound 2 for 9 hours. Nuclear and cytoplasmic fractions were obtained using the NE-PER Nuclear and Cytoplasmic Extraction Reagents (Thermo Fisher Scientific). Controls are discussed in Table 3. Total RNA was isolated total cell lysates, nuclear and cytoplasmic fractions using the RNeasy Plus Mini Kit (Qiagen). RNA samples were then analyzed in the Agilent 2100 Bioanalyzer to determine RNA quality (RIN Score 8 or higher). RNA concentration was determined using the Qubit fluorometer. A TruSeq Stranded Total RNA LT Sample Prep Kit (Illumina) was used to prepare 4 μg of DNAse-treated RNA for poly(A) RNA purification and fragmentation before strand specific cDNA synthesis. cDNA libraries were a-tailed and ligated to indexed adapters. Samples were then PCR amplified and purified with Ampure XP beads and validated with the Agilent 2100 Bioanalyzer. Samples were quantified again by Qubit before being normalized and pooled to be ran on the Illumina HiSeq 2500 using SBS v3 reagents. Raw FASTQ files were analyzed using FastQC v0.11.2 (Andrews S. 2010, FastQC: A Quality Control Tool for High Throughput Sequence Data) and FastQ Screen v0.4.4 (Wingett and Andrews, PubMed PMID: 30254741) and reads were quality-trimmed using fastq-mcf (ea-utils/1.1.2-806). The trimmed reads were mapped to the hg19 assembly of the human genome (the University of California, Santa Cruz, version from igenomes) using STAR v2.5.3a (Dobin et al., PubMed PMID: 23104886). Duplicated reads were marked using Picard tools (v1.127; Broad Institute), the RNA counts generated from FeatureCounts (Liao et al., 2014, Bioinformatics 30(7): 923-30) were TMM normalized, and differential expression analysis was performed using edgeR (Robinson et al., 2010, Bioinformatics 26(1): 139-40). Expression data is represented as TPM (Transcripts per Million). Genes with mRNA TPM values of zero in either the control or experiment conditions were removed from the analysis. Log2 of the average TPM values for the remaining genes of each condition (total, nuclear, and cytoplasmic) were calculated. Only mRNAs with Log2TPM>−1 were considered for further analysis to remove experimental background noise. The TPM readings of the experiment compared with control samples were used to calculate the positive and negative fold changes from their ratios. The differentially expressed mRNAs with fold changes of + or −1.5 FC were subjected to GSEA to obtain the enriched pathways.

### Gene Set Enrichment Analysis (GSEA)

Pathway and network analysis were conducted using Gene Set Enrichment Analysis (GSEA) (Subramanian et at., 2005, PNAS USA 102(43): 15545-50) software and the functional datasets were CP: Canonical pathways from the MSigDB (Liberzon et al., 2015, Cell Syst. 1(6): 417-25; Liberzon et al., 2011, Bioinformatics 27(12): 1739-40).

### Western Blot

Cell lysis was performed in 250 mM Tris HCl pH 6.8, 40% Glycerol, and 8% SDS. Western blot was performed as previously described (Tsai et al., 2013, PLoS pathogens 9(6): e1003460). Antibodies used in this study to detect viral proteins include Influenza A virions (Meridian Life Science B65141G), M1 and M2 (Thermo MA1-082), NA (GeneTex GTX125974), PA (GeneTex GTX118991), PB1 (Santa Cruz sc-17601), PB2 (Santa Cruz sc-17603), and NS1 (a gift from J.A. Richt, National Animal Disease Center, Iowa) (Solorzano et al., 2005, Journal of virology 79(12): 7535-43). Antibodies against cellular proteins include β-actin (Sigma A5441) and UAP56 [Anti-BAT1 (C-TERMINAL antibody produced in rabbit, Millipore SAB1307254). Horseradish peroxidase (HRP)-conjugated secondary antibodies include donkey anti-rabbit, sheep anti-mouse (GE Healthcare NA934V and NA931V, respectively), and donkey anti-goat (Jackson Immunoresearch 705-035003). Quantification of protein band intensity was performed using Image Studio software (LI-COR Imaging). Each protein band was normalized to its corresponding loading control. Values listed below each band represent relative band intensity to its corresponding control.

### Example 1: High-Throughput Screen to Identify Inhibitors of Viral M mRNA Processing and Nuclear Export

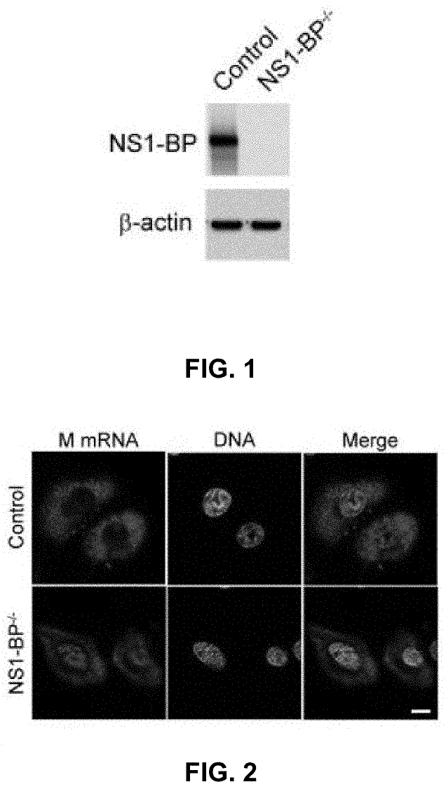

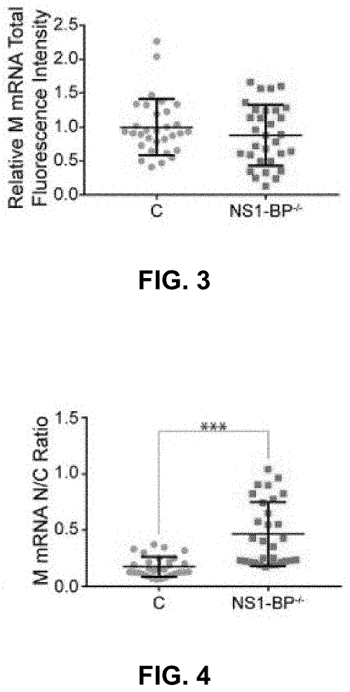

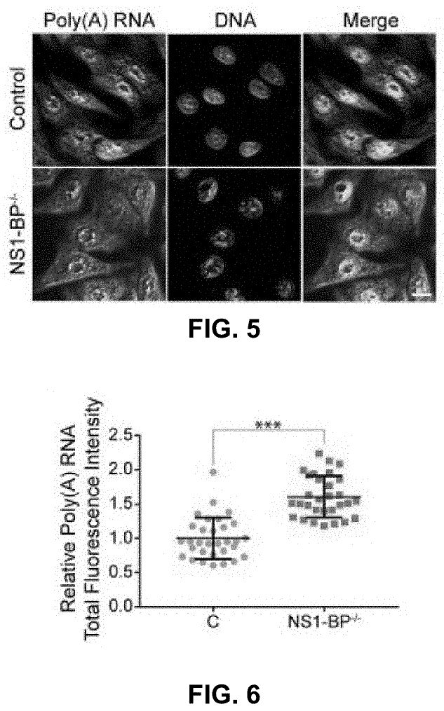

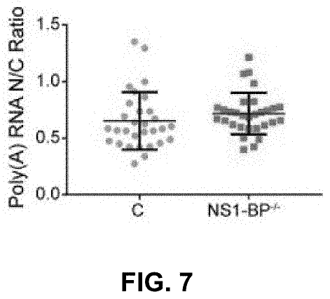

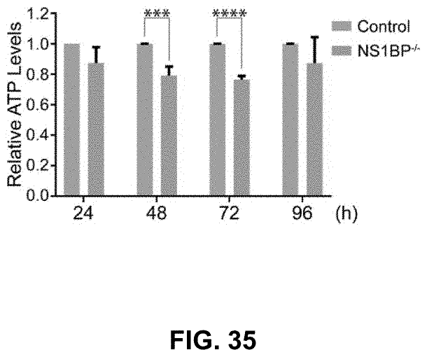

Knockdown of the cellular NS1-BP protein was previously reported to inhibit influenza virus M mRNA splicing and nuclear export through host nuclear speckles (Mor et al., 2016, Nat Microbiol. 1(7): 16069). In this study, NS1-BP was knocked out using the CRISPR/Cas9 system (FIG. 1) and these cells show a slight reduction in growth rate (FIG. 35). wild-type and NS1-BP knockout cells were then subjected to single-molecule RNA fluorescence in situ hybridization (smRNA-FISH) to detect influenza virus M1 mRNA in infected cells (FIGS. 2-4) and oligo-dT in situ hybridization to label bulk cellular poly(A) RNA in the absence of infection (FIGS. 5-7). While viral M1 mRNA nuclear export is substantially inhibited in the absence of NS1-BP (FIGS. 2-4), bulk cellular poly(A) RNA distribution between the nucleus and cytoplasm was not altered in the absence of NS1-BP, but total intracellular levels were increased (FIGS. 5-7). These results indicate that the viral M mRNA uses a distinct mechanism to be exported from the nucleus to the cytoplasm, which is not shared by the bulk of the cellular mRNA. Thus, it was postulated that it should be possible to identify specific inhibitors of this unique mechanism that would impact nuclear export of the influenza virus M RNA without significantly affecting bulk cellular RNA processing and expression.

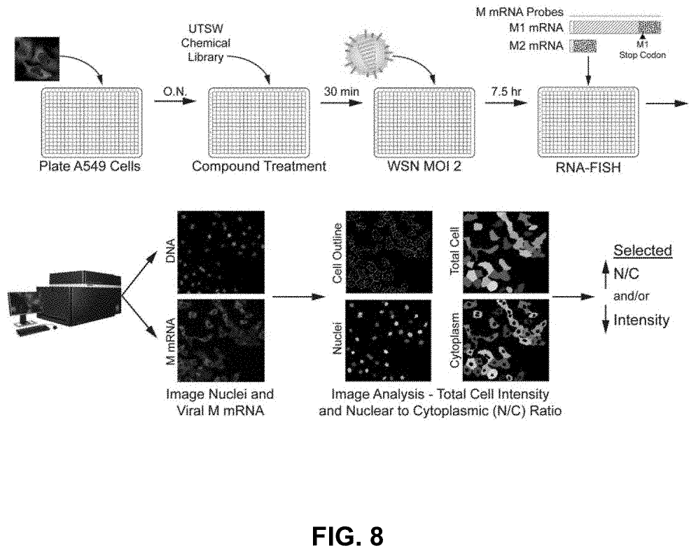

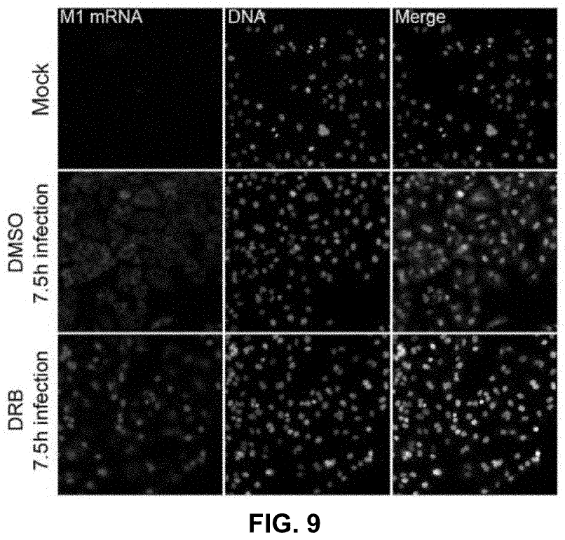

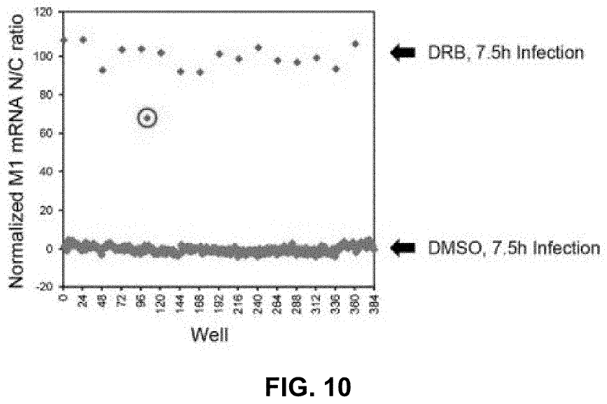

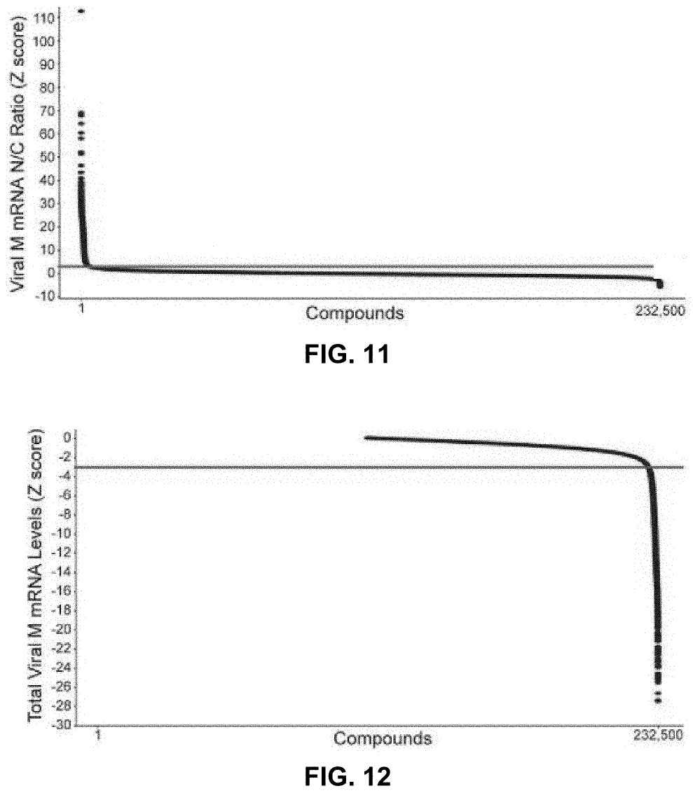

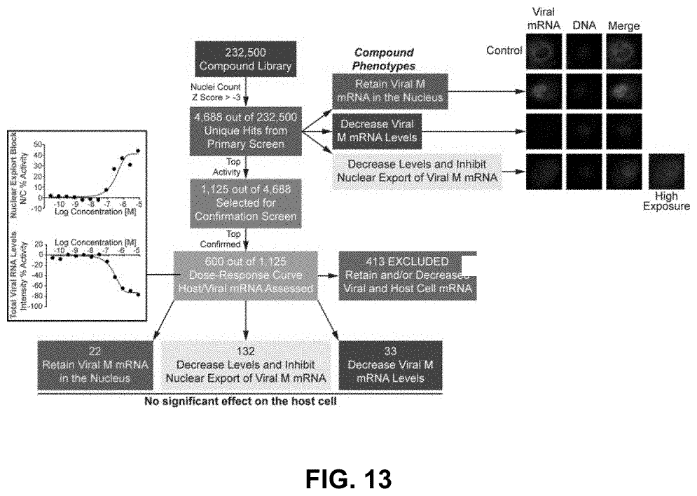

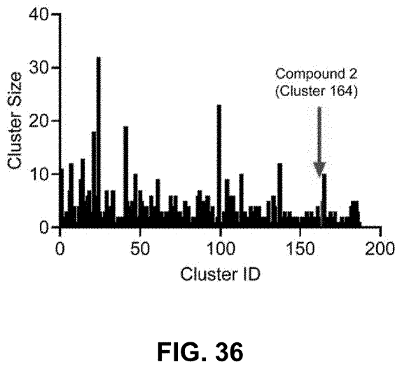

Next, a high-throughput screening was performed to select inhibitors of viral M mRNA processing and nuclear export. The previously reported protocol to visualize the M mRNAs during virus infection (Mor et al., 2016, Nat Microbiol. 1(7): 16069) was adapted and a high-throughput screening assay was designed to identify compounds that alter M mRNA expression and trafficking without significantly compromising bulk cellular poly(A) RNA levels or intracellular distribution. The high throughput screen was performed using a chemical library of 232,500 compounds. As shown in FIG. 8, cells were incubated with compounds and then infected with influenza virus (W SN) for 7.5 h. Cells were then subjected to smRNA-FISH and images were analyzed by quantifying the distribution of fluorescence signal between the nucleus (N) and the cytoplasm (N/C ratio) as well as total cell fluorescence intensity. In a control experiment, N/C ratios were identified for DMSO negative-control and DRB (5,6-dichloro-1-β-D-ribofuranosylbenzimidazole) positive control (FIGS. 9-10), which inhibits cellular processive transcription by RNA polymerase II and also prevents nuclear export of a subset of influenza virus mRNAs, including M mRNA. Compounds with high N/C ratios (Z-score≥3 compared to the robust test population median on each plate),indicating nuclear export block of viral M mRNA (FIG. 11), were selected for follow-up screening. In addition, the screening revealed compounds that selectively decreased viral RNA signal (fluorescence intensity), indicating down-regulation of viral M mRNA levels (Z-score≤−3 compared to the median of the test population on each plate, FIG. 12). Furthermore, compounds that both inhibited viral M mRNA nuclear export and decreased total viral M mRNA levels were also identified (FIG. 13). Compounds that reduced nuclei count were considered cytotoxic (Z-score threshold<−3, See FIG. 13). In total, 4,688 of the 232,500 compounds tested were hits in the primary screen. The 4,688 compounds were clustered based on chemical structure and the 1,125 compounds (824 that inhibited M mRNA nuclear export and 301 that decreased M mRNA levels) with the highest Z-scores from each cluster were chosen for confirmation. The top 600 compounds, including both phenotypic classes, were then subjected to dose response assays to determine their potency. At this stage, poly(A) RNA was also assessed by RNA-FISH to detect potential compound effects on host bulk poly(A) RNA levels or nuclear export. Compounds that altered bulk poly(A) RNA were excluded (AC50<8 μM). Thus, only compounds that blocked viral M mRNA nuclear export or biogenesis and did not substantially affect host bulk mRNA, at non-toxic concentrations, were selected. 413 of the 600 compounds altered bulk poly(A) RNA and were excluded, thus leaving a total of 187 compounds for follow-up studies (FIG. 13). Importantly, these 187 confirmed hits represent 187 structurally diverse clusters, and each cluster contains the top hit and related less active analogs (FIG. 36).

### Example 2: Inhibitors of Viral mRNA Nuclear Export

The identified inhibitors of viral mRNA nuclear export are provided in Table 2. As shown therein, compounds 1-28, corresponding to structural formulas III, II and IV-XXIX, were identified through the above screening as compounds that inhibit nuclear export of influenza virus mRNAs and consequently prevent influenza virus replication at non-toxic concentrations. In Table 2, virus replication was assessed in A549 cells, which were infected with A/WSN/33 at MOI 0.01 in the absence or presence of compound 2 at different concentrations. After 24 h post infection, cells were fixed with 4% formaldehyde for 30 min. Cells were briefly washed with PBS, then permeabilized with 0.1% Triton X-100 in PBS for 15 minutes. Blocking occurred at room temperature for 1 hour with 0.5% BSA in PBS followed by incubation with the NP antibody (HT103) in 0.5% BSA in PBS for 1 h at room temperature. Cells were washed with PBS 2× and incubated with a fluorescently-labeled secondary antibody, alexa-fluor-488 (Invitrogen), in 0.5% BSA in PBS with DAPI for 45 min at room temperature. Two washes with PBS were performed before imaging the cells on a Celigo Image Cytometer. Percent infection was quantified by dividing the number of NP-positive cells by the total number of cells. Cytotoxicity was also performed using the MTT assay (Roche), according to the manufacturer's instructions, concurrent with immunostaining. Replication of A/WSN/33, A/Vietnam/1203/04, and A/Panama/99 were also tested using plaque assays and the IC50s for compound 2 were ˜2-fold less than in NP assays, indicating more inhibition of virus replication when assessing infectious particles.

TABLE 2

| | | | | 24 h | 24 h |
|---|---|---|---|---|---|
| | | WSN | WSN | MTT | MTT |
| | | IC50 | IC90 | CC10 | CC50 |
| Compound | Structure | (μM) | (μM) | (μM) | (μM) |
| 1 | 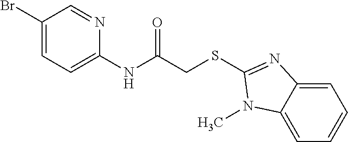 [SMILES: Cn1c(SCC(=O)Nc2ccc(Br)cn2)nc2ccccc21] | 1.886 | 12.055 | 1.417 | >50 |
| | (Structural Formula III) | | | | |
| | CN1C(SCC(═O)NC2═CC═C(Br)C═N2)═NC | | | | |
| | 2═CC═CC═C12 | | | | |
| | (Formula III) | | | | |
| 2 | 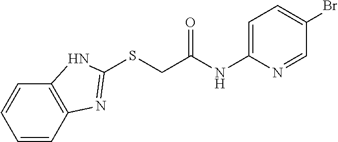 [SMILES: O=C(CSc1nc2ccccc2[nH]1)Nc1ccc(Br)cn1] | 3.184 | 16.904 | 5.839 | >50 |
| | (Structural Formula II) | | | | |
| | BrC1═CC═C(NC(═O)CSC2═NC3═C(N2)C═ | | | | |
| | CC═C3)N═C1 | | | | |
| | (Formula II) | | | | |
| 3 | 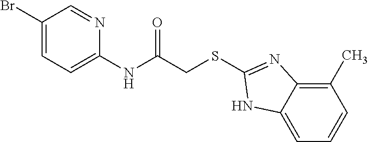 [SMILES: Cc1cccc2[nH]c(SCC(=O)Nc3ccc(Br)cn3)nc12] | 4.942 | 10.035 | 0.683 | >50 |
| | (Structural Formula IV) | | | | |
| | CC1═C2N═C(NC2═CC═C1)SCC(═O)NC1═ | | | | |
| | CC═C(Br)C═N1 | | | | |
| | (Formula IV) | | | | |
| 4 | 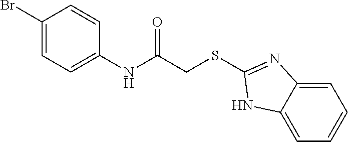 [SMILES: O=C(CSc1nc2ccccc2[nH]1)Nc1ccc(Br)cc1] | >50 | >50 | >50 | >50 |
| | (Structural Formula V) | | | | |
| | BrC1═CC═C(NC(═O)CSC2═NC3═CC═CC═ | | | | |
| | C3N2)C═C1 | | | | |
| | (Formula V) | | | | |
| 5 | 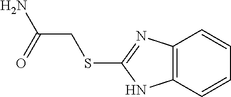 [SMILES: NC(=O)CSc1nc2ccccc2[nH]1] | >50 | >50 | >50 | >50 |
| | (Structural Formula VI) | | | | |
| | NC(═O)CSC1═NC2═C(N1)C═CC═C2 | | | | |
| | (Formula VI) | | | | |
| 6 | 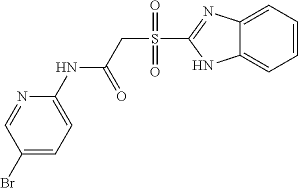 [SMILES: O=C(CS(=O)(=O)c1nc2ccccc2[nH]1)Nc1ccc(Br)cn1] | 22.571 | >50 | 23.361 | >50 |
| | (Structural Formula VII) | | | | |
| | BrC1═CC═C(NC(═O)CS(═O)(═O)C2═NC3 | | | | |
| | ═CC═CC═C3N2)N═C1 | | | | |
| | (Formula VII) | | | | |
| 7 | 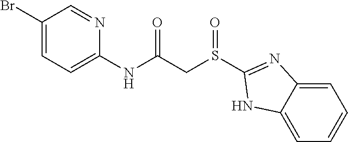 [SMILES: O=C(CS(=O)c1nc2ccccc2[nH]1)Nc1ccc(Br)cn1] | 11.946 | >50 | 31.074 | >50 |
| | (Structural Formula VIII) | | | | |
| | BrC1═CC═C(NC(═O)CS(═O)C2═NC3═CC | | | | |
| | ═CC═C3N2)N═C1 | | | | |
| | (Formula VIII) | | | | |
| 8 | 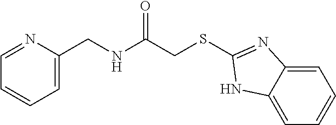 [SMILES: O=C(CSc1nc2ccccc2[nH]1)NCc1ccccn1] | >50 | >50 | >50 | >50 |
| | (Structural Formula IX) | | | | |
| | O═C(CSC1═NC2═CC═CC═C2N1)NCC1═C | | | | |
| | C═CC═N1 | | | | |
| | (Formula IX) | | | | |
| 9 | 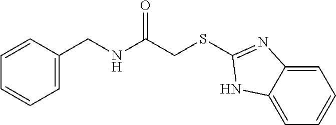 [SMILES: O=C(CSc1nc2ccccc2[nH]1)NCc1ccccc1] | >50 | >50 | >50 | >50 |
| | (Structural Formula X) | | | | |
| | O═C(CSC1═NC2═CC═CC═C2N1)NCC1═C | | | | |
| | C═CC═C1 | | | | |
| | (Formula X) | | | | |
| 10 | 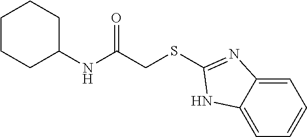 [SMILES: O=C(CSc1nc2ccccc2[nH]1)NC1CCCCC1] | >50 | >50 | >50 | >50 |
| | (Structural Formula XI) | | | | |
| | O═C(CSC1═NC2═CC═CC═C2N1)NC1CCC | | | | |
| | CC1 | | | | |
| | (Formula XI) | | | | |
| 11 | 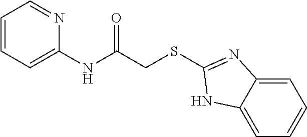 [SMILES: O=C(CSc1nc2ccccc2[nH]1)Nc1ccccn1] | >50 | >50 | >50 | >50 |
| | (Structural Formula XII) | | | | |
| | O═C(CSC1═NC2═CC═CC═C2N1)NC1═CC | | | | |
| | ═CC═N1 | | | | |
| | (Formula XII) | | | | |
| 12 | 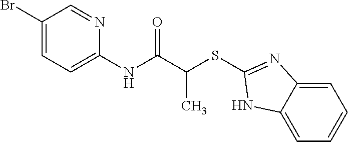 [SMILES: CC(Sc1nc2ccccc2[nH]1)C(=O)Nc1ccc(Br)cn1] | >50 | >50 | >50 | >50 |
| | (Structural Formula XIII) | | | | |
| | CC(SC1═NC2═CC═CC═C2N1)C(═O)NC1═ | | | | |
| | CC═C(Br)C═N1 | | | | |
| | (Formula XIII) | | | | |
| 13 | 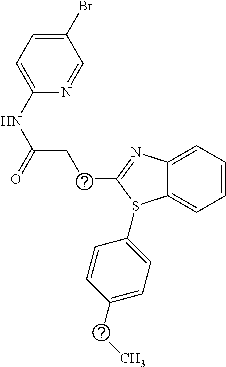 | >50 | >50 | 2.567 | 34.058 |
| | (Structural Formula XIV) | | | | |
| | COC1═CC═C(C═C1)N1C(SCC(═O)NC2═C | | | | |
| | C═C(Br)C═N2)═NC2═CC═CC═C12 | | | | |
| | (Formula XIV) | | | | |
| 14 | 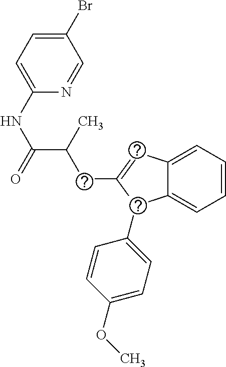 [SMILES: COc1ccc(C2C(CC(C)C(=O)Nc3ccc(Br)cn3)=Cc3ccccc32)cc1] | >50 | >50 | 15.941 | >50 |
| | (Structural Formula XV) | | | | |
| | COC1═CC═C(C═C1)N1C(SC(C)C(═O)NC2 | | | | |
| | ═CC═C(Br)C═N2)═NC2═CC═CC═C12 | | | | |
| | (Formula XV) | | | | |
| 15 | 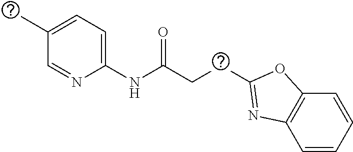 [SMILES: Cc1ccc(NC(=O)CCc2nc3ccccc3o2)nc1] | 30.669 | >50 | >50 | >50 |
| | (Structural Formula XVI) | | | | |
| | BrC1═CC═C(NC(═O)CSC2═NC3═C(O2)C═ | | | | |
| | CC═C3)N═C1 | | | | |
| | (Formula XVI) | | | | |
| 16 | 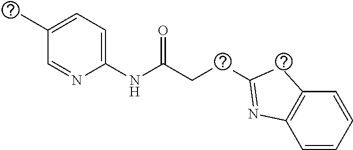 [SMILES: Cc1ccc(NC(=O)CCC2=Nc3ccccc3C2)nc1] | 30.888 | >50 | 7.382 | >50 |
| | (Structural Formula XVII) | | | | |
| | BrC1═CC═C(NC(═O)CSC2═NC3═C(S2)C═ | | | | |
| | CC═C3)N═C1 | | | | |
| | (Formula XVII) | | | | |
| 17 | 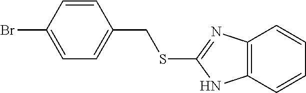 [SMILES: Brc1ccc(CSc2nc3ccccc3[nH]2)cc1] | >50 | >50 | 19.818 | >50 |
| | (Structural Formula XVIII) | | | | |
| | BrC1═CC═C(CSC2═NC3═C(N2)C═CC═C3) | | | | |
| | C═C1 | | | | |
| | (Formula XVIII) | | | | |
| 18 | 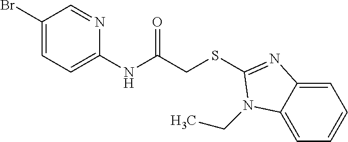 [SMILES: CCn1c(SCC(=O)Nc2ccc(Br)cn2)nc2ccccc21] | 38.371 | >50 | 47.499 | >50 |
| | (Structural Formula XIX) | | | | |
| | CCN1C(SCC(═O)NC2═CC═C(Br)C═N2)═N | | | | |
| | C2═CC═CC═C12 | | | | |
| | (Formula XIX) | | | | |
| 19 | 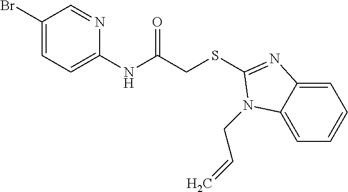 [SMILES: C=CCn1c(SCC(=O)Nc2ccc(Br)cn2)nc2ccccc21] | >50 | >50 | 18.984 | >50 |
| | (Structural Formula XX) | | | | |
| | BrC1═CC═C(NC(═O)CSC2═NC3═CC═CC═ | | | | |
| | C3N2CC═C)N═C1 | | | | |
| | (Formula XX) | | | | |
| 20 | 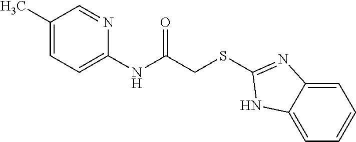 [SMILES: Cc1ccc(NC(=O)CSc2nc3ccccc3[nH]2)nc1] | >50 | >50 | 7.855 | >50 |
| | (Structural Formula XXI) | | | | |
| | CC1═CC═C(NC(═O)CSC2═NC3═CC═CC═ | | | | |
| | C3N2)N═C1 | | | | |
| | (Formula XXI) | | | | |
| 21 | 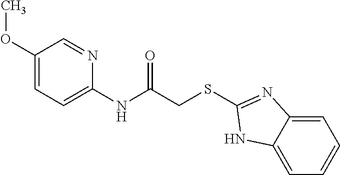 [SMILES: COc1ccc(NC(=O)CSc2nc3ccccc3[nH]2)nc1] | 17.567 | 45.36 | 4.69 | >50 |
| | (Structural Formula XXII) | | | | |
| | COC1═CC═C(NC(═O)CSC2═NC3═CC═CC | | | | |
| | ═C3N2)N═C1 | | | | |
| | (Formula XXII) | | | | |
| 22 | 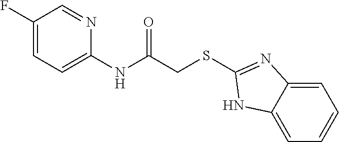 [SMILES: O=C(CSc1nc2ccccc2[nH]1)Nc1ccc(F)cn1] | 43.348 | >50 | 31.688 | >50 |
| | (Structural Formula XXIII) | | | | |
| | FC1═CC═C(NC(═O)CSC2═NC3═CC═CC═ | | | | |
| | C3N2)N═C1 | | | | |
| 23 | 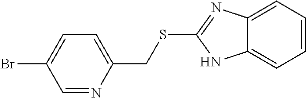 [SMILES: Brc1ccc(CSc2nc3ccccc3[nH]2)nc1] | >50 | >50 | 37.461 | >50 |
| | (Structural Formula XXIV) | | | | |
| | BrC1═CC═C(CSC2═NC3═CC═CC═C3N2) | | | | |
| | N═C1 | | | | |
| | (Formula XXIV) | | | | |
| 24 | 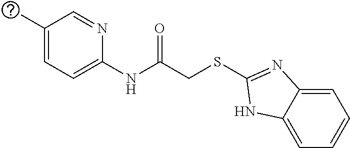 [SMILES: Cc1ccc(NC(=O)CSc2nc3ccccc3[nH]2)nc1] | 8.7 | 24.461 | 5.552 | >50 |
| | (Structural Formula XXV) | | | | |
| | C1C1═CC═C(NC(═O)CSC2═NC3═CC═CC═ | | | | |
| | C3N2)N═C1 | | | | |
| | (Formula XXV) | | | | |
| 25 | 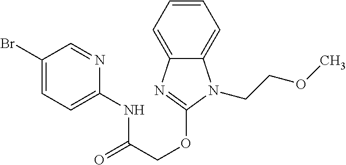 [SMILES: COCCn1c(OCC(=O)Nc2ccc(Br)cn2)nc2ccccc21] | 47.624 | — | >50 | >50 |
| | (Structural Formula XXVI) | | | | |
| | COCCN1C(SCC(═O)NC2═CC═C(Br)C═N2) | | | | |
| | ═NC2═CC═CC═C12 | | | | |
| | (Formula XXVI) | | | | |
| 26 | 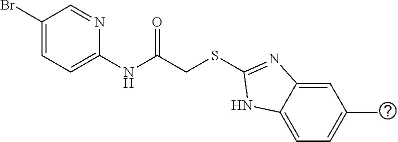 [SMILES: Cc1ccc2[nH]c(SCC(=O)Nc3ccc(Br)cn3)nc2c1] | 25.499 | >50 | 6.797 | 49.675 |
| | (Structural Formula XXVII) | | | | |
| | C1C1═CC═C2NC(SCC(═O)NC3═CC═C(Br) | | | | |
| | C═N3)═NC2═C1 | | | | |
| | (Formula XXVII) | | | | |
| 27 | 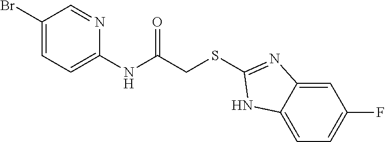 [SMILES: O=C(CSc1nc2cc(F)ccc2[nH]1)Nc1ccc(Br)cn1] | 16.14 | 18.233 | 2.905 | >50 |
| | (Structural Formula XXVIII) | | | | |
| | FC1═CC═C2NC(SCC(═O)NC3═CC═C(Br) | | | | |
| | C═N3)═NC2═C1 | | | | |
| | (Formula XXVIII) | | | | |
| 28 | 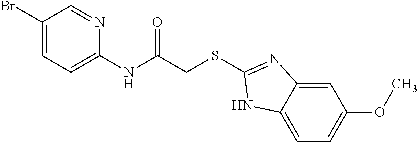 [SMILES: COc1ccc2[nH]c(SCC(=O)Nc3ccc(Br)cn3)nc2c1] | 46.45 | 49.962 | 7.52 | >50 |
| | (Structural Formula XXIX) | | | | |
| | COC1═CC═C2NC(SCC(═O)NC3═CC═C(Br) | | | | |
| | C═N3)═NC2═C1 | | | | |
| | (Formula XXIX) | | | | |

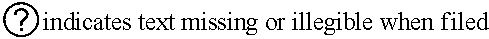

### Example 3: Selective Inhibition of Viral mRNA Nuclear Export

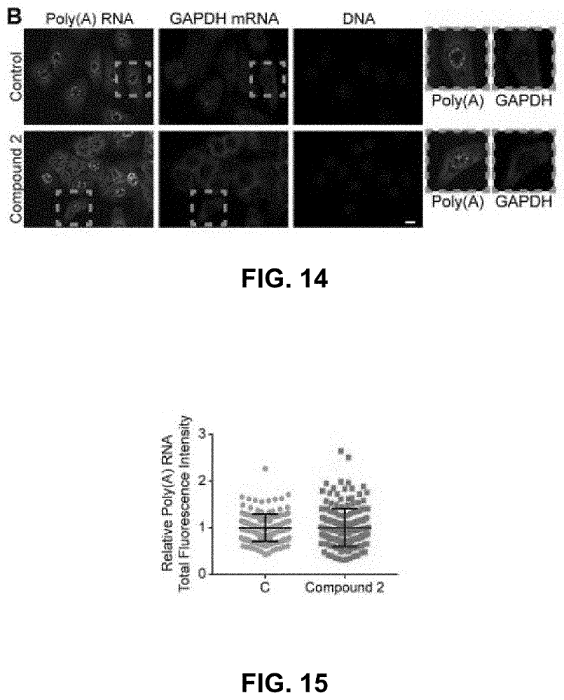

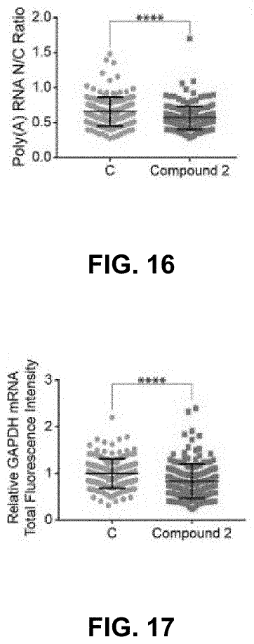

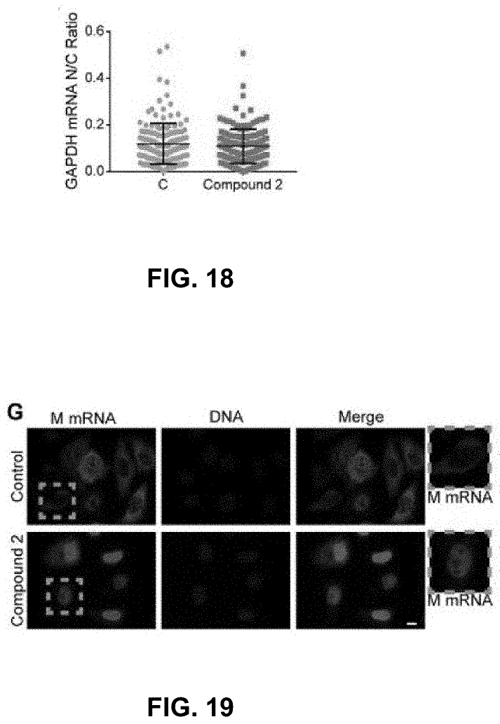

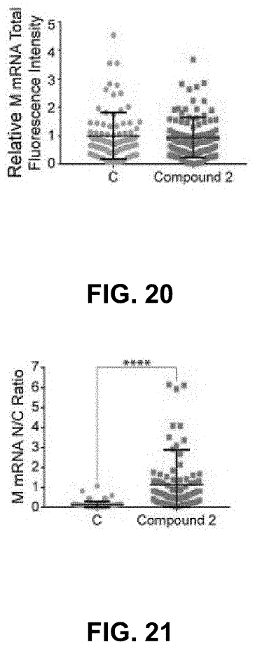

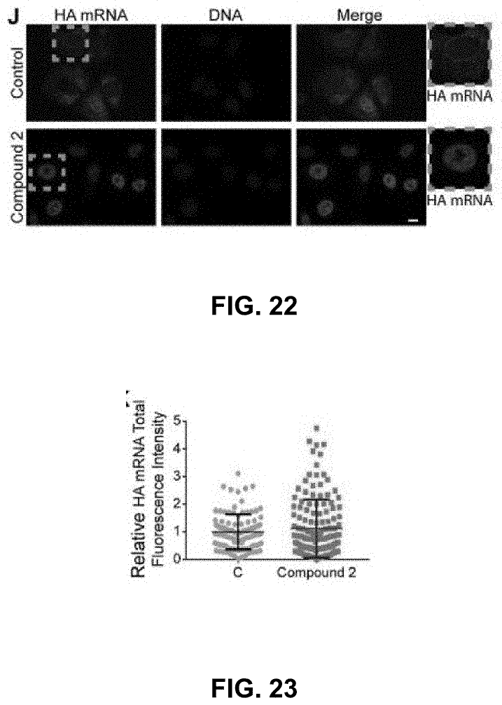

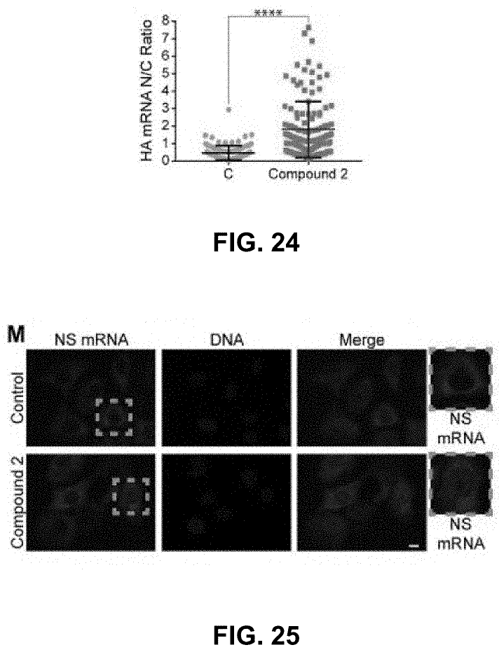

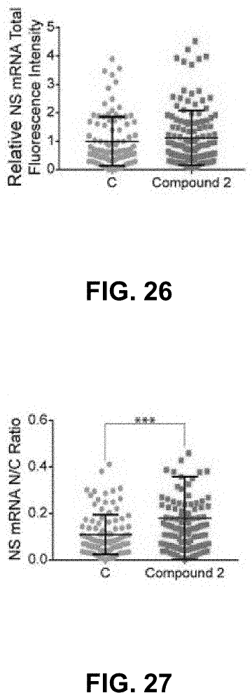

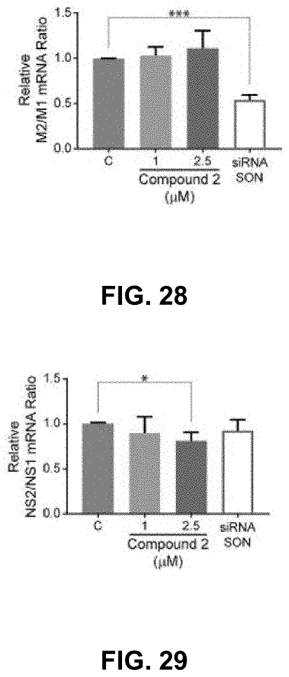

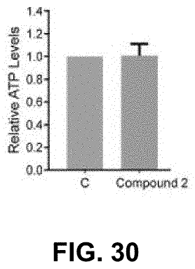

For follow-up studies, compounds with the lowest AC50 in dose-response curves that showed retention of viral M mRNA in the nucleus were first selected by measuring nuclear to cytoplasmic ratios as in FIG. 13. Among the top hits is compound 2, which is a 2-((1H-benzo[d]imidazole-2-yl)thio)-N-(5-bromopyridin-2-yl) acetamide. This compound was re-tested in smRNA-FISH to confirm the intracellular distribution of viral M mRNA and also extended our analysis to other influenza virus mRNAs, including HA and NS, as well as bulk poly(A) RNA and cellular GAPDH mRNA. Image quantification was performed by determining the mRNA fluorescence intensity in whole cells or in the nucleus and cytoplasm, which is expressed as N/C ratios. It was found that compound 2 did not affect the total levels of bulk cellular poly(A) RNA (FIGS. 14-15) and slightly decreased its nuclear to cytoplasmic ratio (FIGS. 14 and 16). The total levels of cellular GAPDH mRNA were also slightly decreased by compound 2 (FIGS. 14 and 17) and its nuclear to cytoplasmic distribution was not affected (FIGS. 14 and 18). Thus, compound 2 slightly promoted cellular poly(A) RNA export. In contrast, compound 2 robustly inhibited nuclear export of viral M mRNA (FIGS. 19-21) and HA mRNA (FIGS. 22-24). Compound 2 did not alter total M mRNA fluorescence intensity (FIGS. 19-20) but induced nuclear retention of M mRNA (FIGS. 19 and 21). A similar result was obtained for the HA mRNA (FIGS. 22-24). The total levels of the NS mRNA were not altered by compound 2 (FIGS. 25-26), but this compound induced a weak nuclear retention of NS mRNA (FIGS. 25 and 27) as compared to the effective inhibition of M and HA mRNAs (FIGS. 19-24). These results highlight differences in requirements for nuclear export of specific influenza virus mRNAs. To assess whether compound 2 had any effect on M1 to M2 splicing, the relative ratio of M2 to M1 mRNAs was quantified in the absence or presence of compound 2 by qPCR. No effect of compound 2 on M1 to M2 splicing was found as opposed to knockdown of the splicing co-factor and nuclear speckle assembly factor SON, which was used as a positive control (SON promotes M1 to M2 splicing at nuclear speckles; See FIG. 28). In addition, compound 2 only slightly inhibited NS1 to NS2 splicing (FIG. 29). Cellular ATP levels were also assessed as a surrogate for cytotoxicity and showed no significant change in ATP levels (FIG. 30). Thus, these findings indicate that compound 2 robustly targets nuclear export of a subset of mRNAs at non-toxic concentrations.

### Example 4: Effect of Compound 2 on M mRNA Nuclear Export Pathway

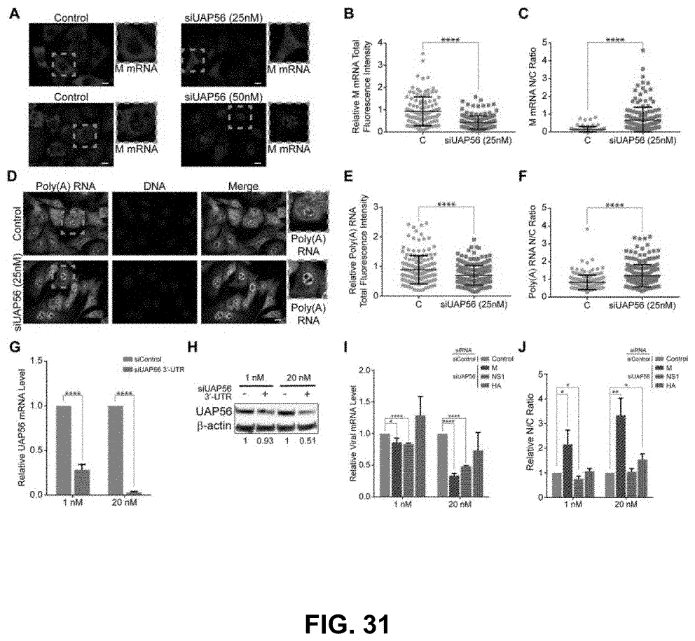

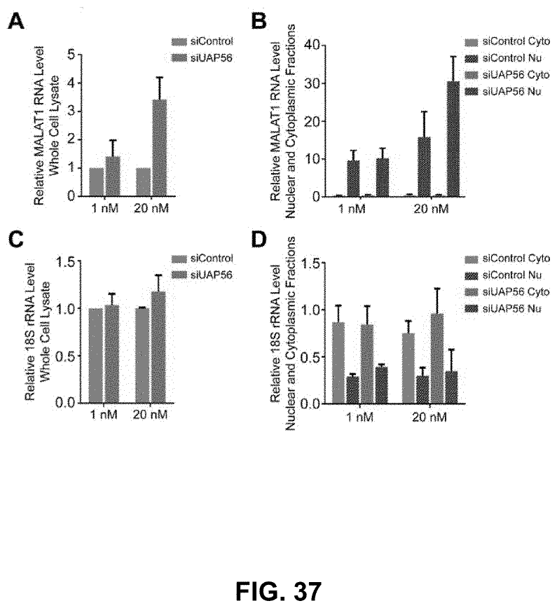

The effect of compound 2 on the M mRNA nuclear export pathway was subsequently tested. It was previously shown that that M mRNA nuclear export is inhibited by knockdown of the mRNA export factor UAP56 (Mor et al., 2016, Nat Microbiol. 1(7): 16069; Wisskirchen et al., 2011, Journal of Virology 85(17): 8646-55; Read et al., 2010, The Journal of General Virology 91(Pt 5): 1290-301). This effect is also shown here with increasing concentrations of siRNAs that target UAP56 (FIGS. 31A-31C), emphasizing that UAP56 is critical for M mRNA nuclear export. When UAP56 mRNA was knocked down with 25 nM siRNA, a slight reduction in total levels of bulk poly(A) RNA (FIGS. 31D-31E) and partial inhibition of bulk cellular poly(A) RNA nuclear export (FIGS. 31D-31F) were detected. The total levels and intracellular distribution of viral M, HA, and NS1 mRNAs upon depletion of UAP56 were then analyzed with low concentrations of siRNA, which reduced UAP56 mRNA and protein levels in a dose-dependent manner (FIGS. 31G-31H). Upon UAP56 depletion, purified RNA from total cell extracts, nuclear and cytoplasmic fractions were subjected to qPCR (controls for cell fractionation are shown in FIG. 37). Knockdown of UAP56 with 1 nM siRNA only slightly reduced the total levels of M and NS1 mRNAs and did not affect the levels of HA mRNA (FIG. 31I). However, this level of UAP56 down-regulation was sufficient to significantly block M mRNA in the nucleus while the intracellular distribution of NS1 and HA mRNAs were not affected (FIG. 31J). When the siRNA concentration targeting UAP56 was increased to 20 nM, total levels of M and NS1 mRNAs were reduced but HA mRNA level was not altered (FIG. 31I). Nevertheless, M mRNA nuclear export was further blocked, HA mRNA export is also inhibited, and no effect was observed with NS1 mRNA (FIG. 31J). This preferential blockage of M and HA mRNAs by partial depletion of UAP56 is similar to compound 2 effect on viral mRNA export (FIGS. 19-27). A similar pattern of preferential viral mRNA export upon UAP56 depletion has been previously described, but high levels of UAP56 siRNA have been shown to inhibit NS1 mRNA export.

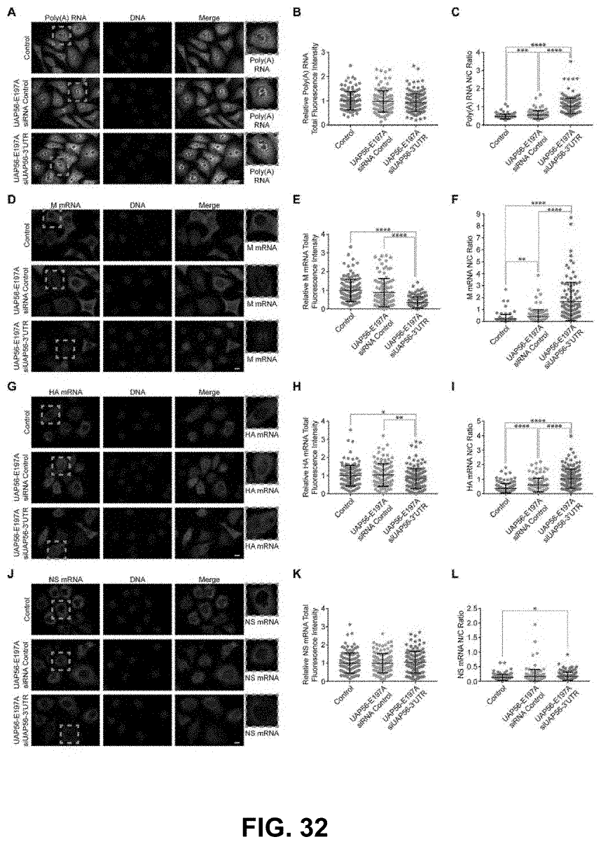

To further corroborate these data, the effect of a catalytically inactive mutant of UAP56 (E197A) was tested on nuclear export of viral M, HA, NS1, and poly(A) RNA. Cells stably expressing UAP56 (E197A) were generated as previously reported (Hondele et al., 2019, Nature 573(7772): 144-8). These cells were treated with control siRNA or with siRNA that targets the 3′UTR of UAP56—this siRNA depletes endogenous UAP56 and not UAP56 mutant. The efficiency of this siRNA is shown in FIGS. 31G and H. Control cells and UAP56 (E197A) mutant cells were thensubjected to RNA-FISH to label poly(A) RNA (FIGS. 31A-31C) or infected with WSN followed by smRNA-FISH to detect M, HA, and NS1 mRNAs (FIGS. 32D-32L) followed by fluorescence microscopy. In the UAP56 mutant cells treated with siRNA control, the total levels of these mRNAs are not altered while nuclear export of M and HA mRNAs is preferentially blocked, poly(A) RNA export is slightly inhibited, and NS1 mRNA export is not altered (FIG. 32A-32L). When these mutant UAP56 cells were then treated with siRNA against endogenous UAP56, the total levels of M and HA mRNAs were reduced (FIGS. 32E and 32H) and the levels of poly(A) RNA and NS1 mRNA were not altered (FIGS. 32B and 32K). On the other hand, nuclear export of M mRNA was severely blocked, poly(A) RNA and HA mRNA export was also inhibited, and NS1 mRNA was only slightly altered (FIGS. 32C-32L). Taken together, these results show that compound 2 phenocopies partial down-regulation of UAP56 activity as shown by either depleting UAP56 with low levels of siRNA (FIGS. 31G, 31H and 31J) or by expressing UAP56 mutant in the presence of endogenous UAP56 (UAP56-E197A+siRNA control) (FIG. 32A-32L). Since UAP56 is a critical mRNA export factor for viral M mRNA, these results further corroborate the screening strategy to identify inhibitors of the M mRNA nuclear export such as compound 2. Additionally, the differential effect of down-regulating UAP56 activity on nuclear export of certain viral mRNAs further emphasize the concept of preferential usage of specific mRNA export factors or adaptors by a subset of mRNAs.

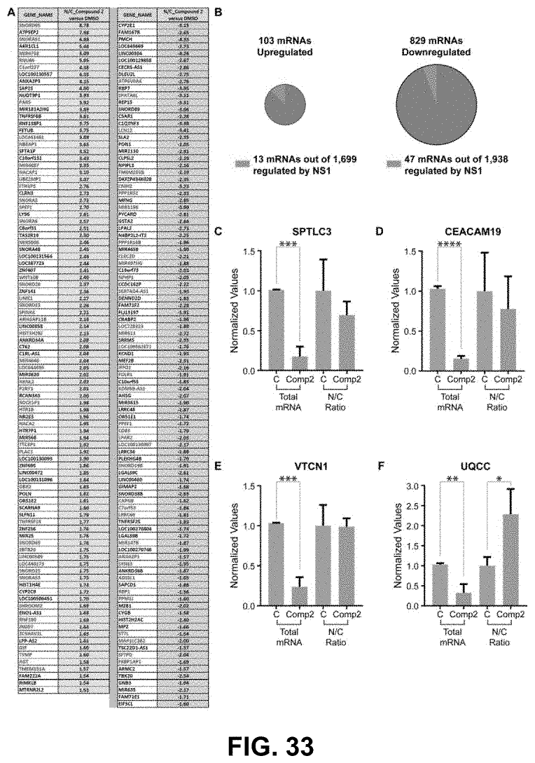

To quantitatively assess a potential impact of compound 2 on a subset of cellular RNAs and determine their identity, RNA-sequencing (RNA-seq) analysis was performed of purified poly(A) RNA obtained from whole cells, nuclear fractions, and cytoplasmic fractions either treated with DMSO (control) or with 2.5 μM of compound 2. As expected, RNAs that are known to be retained in nucleus, such as MALAT1, are primarilynuclear, and mRNAs that are distributed in the nucleus and cytoplasm, such as GAPDH mRNA, are shown in both compartments. A total of 19,799 unique RNAs were sequenced and the cutoff was 1.5-fold change to be considered differentially expressed in the presence of compound 2. It was shown that compound 2 altered the nuclear to cytoplasmic distribution of a small subset of cellular RNAs, including mRNAs and non-coding RNAs (FIG. 33A). Among the non-coding RNAs were small nucleolar RNAs (snoRNAs), miRNAs, and long non-coding RNAs. While snoRNAs are not polyadenylated, pre-snoRNA polyadenylation has been shown to link different steps of snoRNA processing. Similarly, pre-miRNAs are polyadenylated and some long non-coding RNAs also have poly(A) tails, explaining their presence in our poly(A) RNA selection. It was found that the nuclear to cytoplasmic distribution of 194 mRNAs were altered upon compound 2 treatment (FIG. 33A). Among these mRNAs, 96 were preferentially retained in the nucleus (high nuclear/cytoplasmic ratio) (FIG. 33A, yellow) and 98 were more cytoplasmic compared to control cells (FIG. 33A, blue). Within the mRNAs blocked in the nucleus, 48 out of the 96 mRNAs were not altered at their total levels (FIG. 33A, gene name marked in red) indicating nuclear export block similar to the viral M and HA mRNAs upon compound 2 treatment (FIGS. 19-24) suggesting enhanced nuclear export (FIG. 33A, gene name marked in red). Regarding the additional mRNAs that had both altered total levels and nuclear to cytoplasmic ratios, the regulation may or may not involve nuclear transport as other RNA processing steps could be also compromised, which is a topic for future investigation. This RNAseq analysis also revealed the subset of mRNAs up-regulated (103 mRNAs) and down-regulated by compound 2 (829 mRNAs) (FIG. 33B). Among these groups, a small number of mRNAs (13 up-regulated and 47 down-regulated) are also known to be regulated by the viral NS1 protein, as shown in infections performed with WSN compared to WSNANS1. In the category of down-regulated mRNAs, gene set enrichment analysis (GSEA) showed tyrosine metabolism altered by compound 2 (p-value=2.23×10⁻⁵ and a FDR q-value=4.98×10⁻287²). FIGS. 33C-33F show examples of selected mRNAs whose total levels as well as nuclear and cytoplasmic distribution were assessed by qPCR and were consistent with our RNAseq results. Thus, these results indicate an effect of compound 2 on a subset of RNAs and not on bulk poly(A) RNA.

### Example 5: Effects of Compound 2 on Replication of Diverse Influenza Viruses

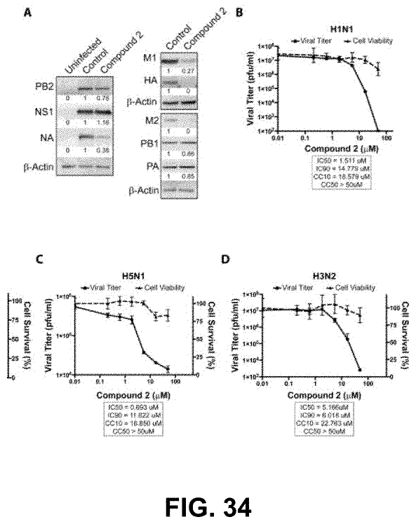

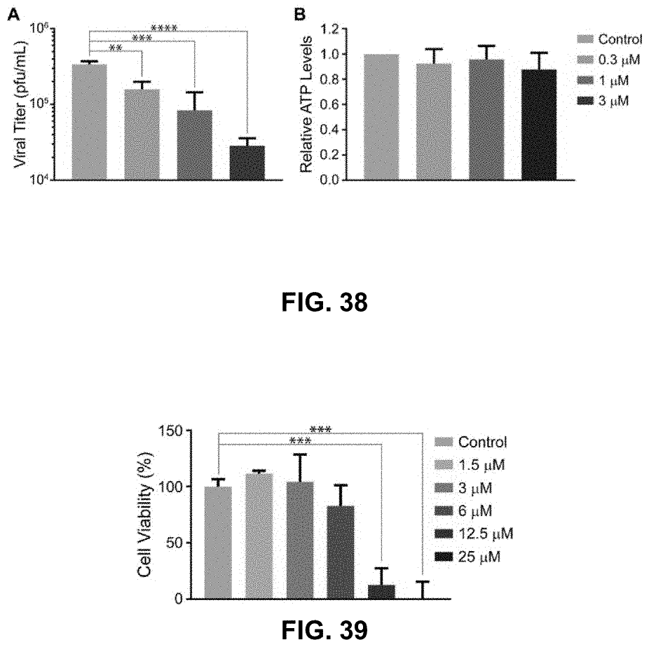

Since nuclear export of key viral mRNAs is blocked by compound 2 and given that these mRNAs encode critical proteins for the virus life cycle, it is expected that viral protein levels and replication would be altered by this compound. Indeed, there is a decrease in the levels of the viral M1 and M2 proteins as well as NA and HA proteins upon 2.5 μM compound treatment (FIG. 34A). Compound 2 was then tested for inhibition of virus replication and cytotoxicity. As expected, compound 2 inhibited replication of diverse influenza A virus strains at concentrations in which it did not significantly alter cell viability (FIGS. 34B-34D). Compound 2 also inhibited viral replication in primary human bronchial epithelial cells (FIGS. 38A-38B). Another compound from our chemical library, ivermectin, is shown as positive control for cytotoxicity at the concentrations used for compound 2 (FIG. 39). In summary, compound 2 preferentially inhibited nuclear export of a subset of mRNAs and further revealed specific requirements for nuclear export of a subset of viral and cellular mRNAs.

### Discussion

While antiviral treatments currently approved for clinical use target viral proteins directly, the presently disclosed compounds target cellular proteins without causing cytotoxicity. Because current antiviral compositions target viral proteins, such treatments have an increased probability of developing strains resistant to these antiviral compositions. In contrast, the presently disclosed compounds are effective against a variety of influenza viral strains as they target cellular proteins thus it is more difficult for antiviral resistant mutations to develop.

With the lack of robust and diverse medical interventions available, multiple antiviral strategies are needed to provide additional therapeutic options for influenza infections. One strategy is to identify viral-host interactions that can be targeted without compromising major host cellular functions. As the virus enters the host cell via endocytosis, the viral M2 ion channel on the viral membrane acidifies the interior of the virus particle. This enables viral uncoating and subsequent release of the viral genome into the host cytoplasm upon fusion of the viral and endosomal membranes. As the eight unique vRNPs enter the host cell nucleus, transcription initiates and 2 of the 8 viral mRNAs undergo alternative splicing. It is the alternative splicing event of the viral M1 mRNA into the viral M2 mRNA that generates the viral M2 protein that is key for viral entry. The M2 protein is also important for viral budding and inhibition of autophagy. M1 mRNA also encodes the M1 protein, which has key functions in viral intracellular trafficking and as a structural component of the infectious virions.

Based on knowledge of viral M mRNA trafficking through host nuclear speckles for splicing and nuclear export, a high-throughput screening strategy was designed that led to the identification of small molecules that interfered with specific steps of this pathway. The image-based chemical screening, which used single-molecule RNA-FISH, identified three classes of inhibitors that either decreased viral M mRNA levels (class 1), or blocked it in the nucleus (class 2), or both (class 3). Our primary HTS assay proved to be quite robust, as exemplified by an average Z′ value of 0.63 for the N/C ratio when comparing the DMSO (vehicle) control to a positive control, DRB. To ensure that all of the chemical space identified by the screen was sampled, the initial set of hits was clustered into chemical series for compounds that decreased the M mRNA fluorescence intensity (552 clusters, intensity reduced >25%) and for compounds that decreased the N/C ratio (˜1300 clusters, N/C ratio >25%). Cluster representatives were then selected from both groups as described above. Hit confirmation studies identified ˜600 compounds that fell into the three phenotypic classes described above. These compounds were subsequently reviewed for chemical attractiveness (e.g. absence of problematic substructures or PAINS, synthetic tractability, etc.). An inhibitor that preferentially prevented nuclear export of a subset of viral mRNAs (class 2) was further tested, resulting in accumulation in the nucleoplasm. Since this small molecule (and others like it identified by the screen) did not substantially alter bulk cellular mRNA levels or their intracellular distribution and were not cytotoxic at active concentrations, they may serve as leads for potential antiviral therapy. Therefore, these data revealed a window of opportunity to target a pathway that processes a subset of viral and cellular mRNAs. In addition, compound 2's differential nuclear export inhibition of viral mRNAs and cellular mRNAs demonstrates specific requirements within the mRNA export machinery for nuclear export and provides a tool to distinguish these pathways in future studies.

The differential effect of compound 2 on viral M mRNA nuclear export, phenocopying down-regulation of UAP56 activity, further corroborates its action on the UAP56-NXF1-mediated mRNA export pathway. This would be predicted based on the screening strategy presented here. UAP56 is known to recruit the mRNA export factor Aly/REF to the mRNA, which then binds the mRNA export receptor NXF1′NXT1. This interaction displaces UAP56 from the mRNA and NXF1′NXT1 then docks the mRNP to the nuclear pore complex for export into the cytoplasm. Prior to docking at the nuclear pore complex, the M mRNA is spliced at nuclear speckles and then exported to the nucleoplasm for translocation through the nuclear pore complex. UAP56 is localized at nuclear speckles and in the nucleoplasm and is required for exit of M mRNA from nuclear speckles to the nucleoplasm as previously shown (Mor et al., 2016, Nat Microbiol. 1(7): 16069). The localization and export function of UAP56 in the nucleoplasm and at nuclear speckles may involve different factors/adaptors. In contrast to M mRNA and a subset of cellular mRNAs whose splicing and/or export occur at nuclear speckles, most cellular mRNAs are spliced in the nucleoplasm prior to being exported from the nucleus. Compound 2 targets the viral M mRNA nuclear export without affecting its splicing at nuclear speckles. Therefore, it is likely that this small molecule is targeting a step between nuclear speckles and the nuclear pore complex, resulting in the accumulation of viral M mRNA throughout the nucleoplasm. Since bulk cellular mRNAs were not substantially affected by the compound at a concentration that it robustly inhibited M and HA mRNA nuclear export, it is possible that this compound is specifically targeting a step or location that affects a subset of cellular mRNAs. In fact, RNAseq analysis shows effect of compound 2 on nuclear export and total levels of a subset of cellular RNAs. This is consistent with the data in which partial depletion of UAP56 or expression of a UAP56 mutant in the catalytic domain in the presence of endogenous UAP56 preferentially blocked viral M and HA mRNA nuclear export without substantially altering NS1 mRNA or bulk cellular mRNAs. These differential effects by partially decreasing the levels of an mRNA export factor reveal a window of opportunity to therapeutically target the mRNA export machinery without inducing major cytotoxicity to the host cell.

Among the subset of cellular mRNAs whose total levels are up-regulated or down-regulated by compound 2 without changes in intracellular distribution, are a few mRNAs known to be regulated by the viral NS1 protein. In the category of up-regulated mRNAs are members of the Type-I interferon response system, including IFIT1 and IRF7 (Diamond, 2014, Cytokine Growth Factor Rev. 25(5): 543-50). IFN response is known to be suppressed by the NS1 protein therefore both IFIT1 and IRF7 mRNAs are up-regulated in cells infected with the influenza virus lacking NS1 protein. Regarding the down-regulated mRNAs, which were enriched in mRNAs that encode proteins involved in tyrosine metabolism, it is possible that the decrease in tyrosine metabolism inhibits virus replication. Tyrosine is a critical amino acid for viral proteins, such as tyrosine 132 phosphorylation of M1 protein which controls its nuclear import and virus replication. Additionally, virus replication is blocked by receptor tyrosine kinase inhibitors. Furthermore, 47 mRNAs in this down-regulated category are also regulated by NS1. Together, these data suggest that inhibition of influenza virus replication by compound 2 may be a combinatory effect of inhibition of viral mRNA export and induction of antiviral response which, at least in part, involves the Type-I interferon system.

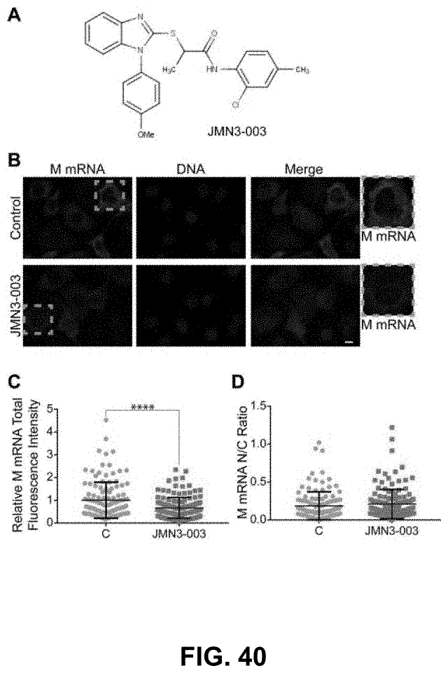

Compound 2 is an alkylated mercaptobenzimidazole featuring an aminopyridine amide. No biological activities have been attributed to this compound previously. However, a structurally related series of N-aryl mercaptobenzimidazoles have been described as inhibitors of influenza viruses and myxoviruses. It was shown that the most potent compound of this series had no effect on M mRNA nuclear export (FIG. 40), indicating that this series operates through a distinct mechanism(s). Accordingly, compound 2 represents an attractive starting point for additional drug discovery efforts. In addition, the screen presented here yielded compounds with various phenotypes—inhibitors of viral M mRNA biogenesis, processing, and/or nuclear export—thus, this strategy expands the landscape for targeting influenza virus at multiple steps of the virus M mRNA intranuclear pathway. As robust viral therapy will likely rely on combination of drugs, this strategy provides multiple leads for drug development. This combinatorial process also contributes to enhance efficacy against diverse viral strains as these compounds may differentially target influenza virus strains. These small molecules are also valuable tools for further understanding new cell biology. They will likely uncover critical regulatory steps and novel factors involved in a yet understudied viral mRNA processing and export pathway.

Although the present invention and its advantages have been described in detail, it should be understood that various changes, substitutions and alterations can be made herein without departing from the spirit and scope of the invention as defined in the appended claims.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A compound of Formula I or a pharmaceutically acceptable salt, solvate, hydrate, prodrug, or derivative thereof: 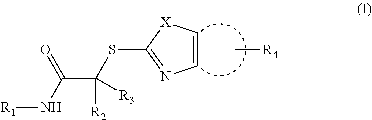
wherein R₁ is an unsubstituted or substituted heteroaryl; R₂ and R₃ are either the same or different and are selected from H or alkyl; X is selected from NH, NR₅, O, and S; R₄ is appended to an optional ring as part of a benzo-fused heteroaryl and is selected from H, alkyl or halogen; and R₅ is an alkyl or aryl.

2. The compound of claim 1, wherein Xis NR₅.

3. The compound of claim 2, wherein R5 is selected from the group consisting of methyl, ethyl, allyl, an alkoxyl, and an alkoxy substituted aryl.

4. The compound of claim 1, wherein X is NH.

5. The compound of claim 1, wherein R1 is a substituted or unsubstituted pyridyl.

6. The compound of claim 1, wherein R1 is unsubstituted or is methyl-, alkoxyl-, or halo-substituted.

7. The compound of claim 5, wherein R1 is a halo-substituted pyridyl.

8. The compound of claim 7, wherein R1 is a chloro-, fluoro-, or bromo-substituted heteroaryl.

9. The compound of claim 1, wherein R2 and R3 are each hydrogen.

10. The compound of claim 1, wherein R4 is hydrogen, a halo, or an alkoxy.

11. The compound of claim 10, wherein R4 is hydrogen, chloro, fluoro, or methoxy.

12. The compound of claim 1, wherein the compound of Formula I is selected from the group consisting of: 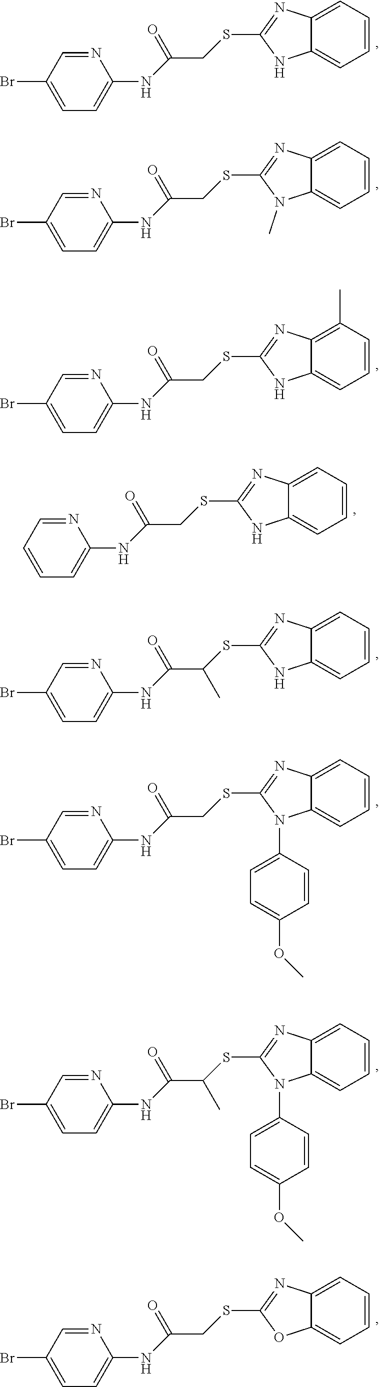 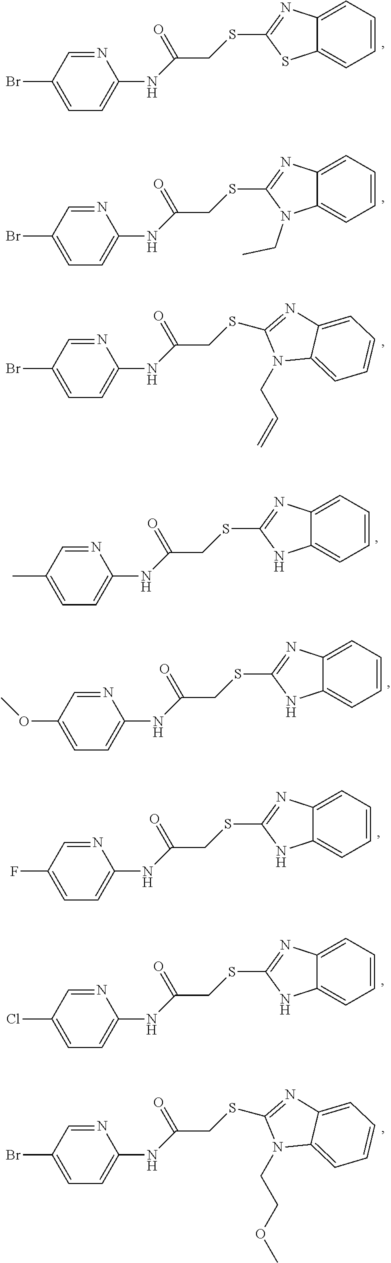 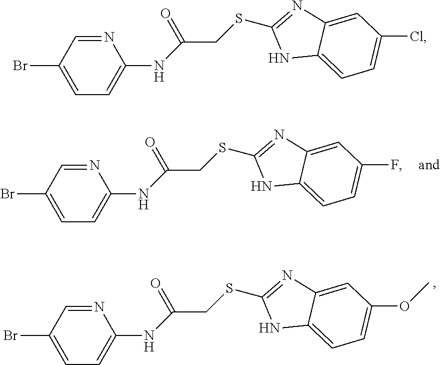 [SMILES: O=C(CSc1nc2cc(Cl)ccc2[nH]1)Nc1ccc(Br)cn1 ; O=C(CSc1nc2cc(F)ccc2[nH]1)Nc1ccc(Br)cn1 ; COc1ccc2[nH]c(SCC(=O)Nc3ccc(Br)cn3)nc2c1]
or a pharmaceutically acceptable salt, solvate, hydrate, prodrug, or derivative thereof.

13. The compound of claim 12, wherein the compound of Formula I is 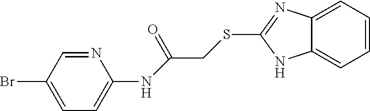 [SMILES: O=C(CSc1nc2ccccc2[nH]1)Nc1ccc(Br)cn1]
or a pharmaceutically acceptable salt, solvate, hydrate, prodrug, or derivative thereof.

14. A compound comprising a structural formula selected from the group consisting of: 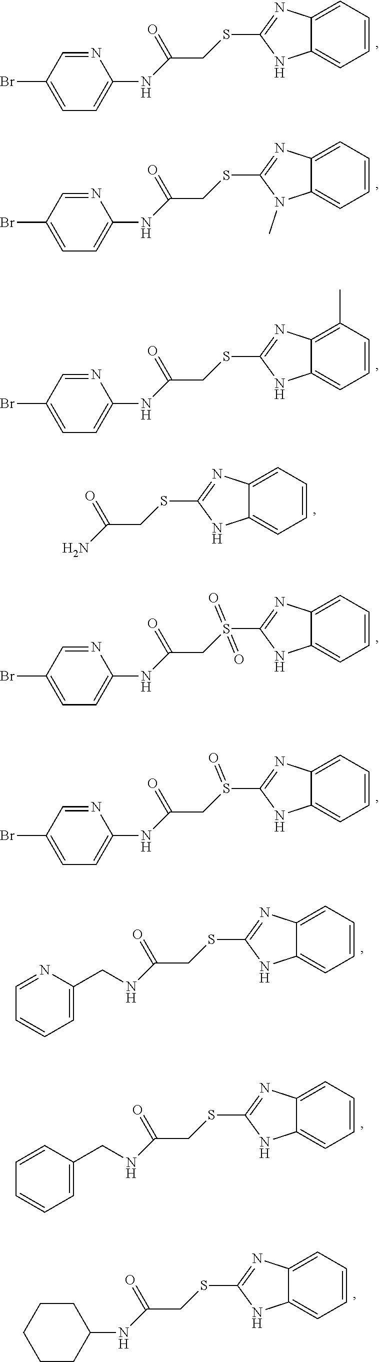 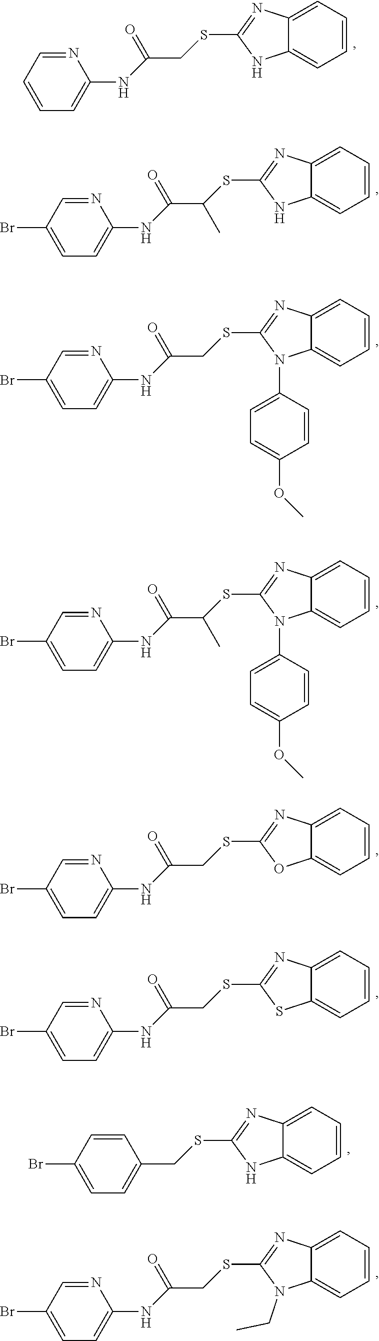 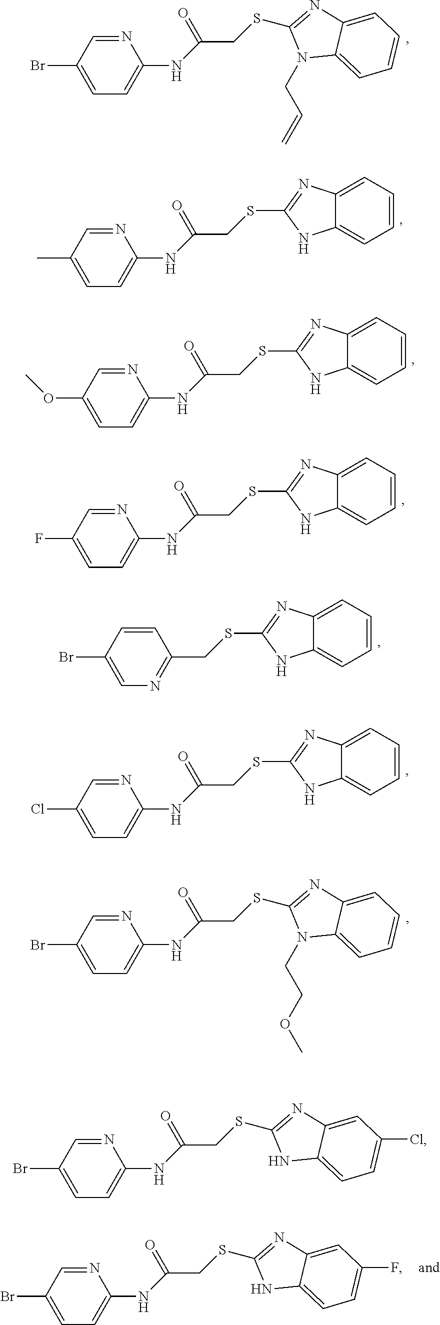 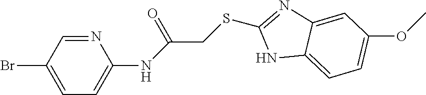 [SMILES: COc1ccc2[nH]c(SCC(=O)Nc3ccc(Br)cn3)nc2c1]
or a pharmaceutically acceptable salt, solvate, hydrate, prodrug, or derivative thereof.


SEQUENCE LISTING (75 sequences)

SEQ ID NO: 1 - DNA, 20 bp, Artificial Sequence
  Synthesized Sequence
catattgtgtctgcatctgt

SEQ ID NO: 2 - DNA, 20 bp, Artificial Sequence
  Synthesized Sequence
ttgagttgttcgcatggtag

SEQ ID NO: 3 - DNA, 20 bp, Artificial Sequence
  Synthesized Sequence
gccacattcttctcgaatat

SEQ ID NO: 4 - DNA, 20 bp, Artificial Sequence
  Synthesized Sequence
gtcttcgagcaggttaacag

SEQ ID NO: 5 - DNA, 20 bp, Artificial Sequence
  Synthesized Sequence
ttacatagtttcccgttgtg

SEQ ID NO: 6 - DNA, 20 bp, Artificial Sequence
  Synthesized Sequence
caattgtagtggggctattc

SEQ ID NO: 7 - DNA, 20 bp, Artificial Sequence
  Synthesized Sequence
catccggtgatgttacattt

SEQ ID NO: 8 - DNA, 20 bp, Artificial Sequence
  Synthesized Sequence
tgagtcgcattctggatttc

SEQ ID NO: 9 - DNA, 20 bp, Artificial Sequence
  Synthesized Sequence
cattctcagagtttggtgtt

SEQ ID NO: 10 - DNA, 20 bp, Artificial Sequence
  Synthesized Sequence
tcagttcctcatagtcgatg

SEQ ID NO: 11 - DNA, 20 bp, Artificial Sequence
  Synthesized Sequence
gatactgagctcaattgctc

SEQ ID NO: 12 - DNA, 20 bp, Artificial Sequence
  Synthesized Sequence
ccatgaactttccttgggaa

SEQ ID NO: 13 - DNA, 20 bp, Artificial Sequence
  Synthesized Sequence
gagcatgatactgttactcc

SEQ ID NO: 14 - DNA, 20 bp, Artificial Sequence
  Synthesized Sequence
gtaaaaactgctttttcccc

SEQ ID NO: 15 - DNA, 20 bp, Artificial Sequence
  Synthesized Sequence
ttcgtcagccatagcaaatt

SEQ ID NO: 16 - DNA, 20 bp, Artificial Sequence
  Synthesized Sequence
aattggtcagctttgggtat

SEQ ID NO: 17 - DNA, 20 bp, Artificial Sequence
  Synthesized Sequence
tttccctttattgttcacat

SEQ ID NO: 18 - DNA, 20 bp, Artificial Sequence
  Synthesized Sequence
tgatgaacaccccatagtac

SEQ ID NO: 19 - DNA, 20 bp, Artificial Sequence
  Synthesized Sequence
gggtgaatctcctgttataa

SEQ ID NO: 20 - DNA, 20 bp, Artificial Sequence
  Synthesized Sequence
cccatgttgatcttttactt

SEQ ID NO: 21 - DNA, 20 bp, Artificial Sequence
  Synthesized Sequence
gcaaggtccagtaatagttc

SEQ ID NO: 22 - DNA, 20 bp, Artificial Sequence
  Synthesized Sequence
tattagattaccagttgcct

SEQ ID NO: 23 - DNA, 20 bp, Artificial Sequence
  Synthesized Sequence
tcagtgcgaaagcataccat

SEQ ID NO: 24 - DNA, 20 bp, Artificial Sequence
  Synthesized Sequence
tgatgatgccggactcaaac

SEQ ID NO: 25 - DNA, 20 bp, Artificial Sequence
  Synthesized Sequence
tcatgcattgacgcgtttga

SEQ ID NO: 26 - DNA, 20 bp, Artificial Sequence
  Synthesized Sequence
gtgtttgacacttcgtgtta

SEQ ID NO: 27 - DNA, 20 bp, Artificial Sequence
  Synthesized Sequence
gattgctgtttatagatccc

SEQ ID NO: 28 - DNA, 20 bp, Artificial Sequence
  Synthesized Sequence
gactgggtgtatattctgga

SEQ ID NO: 29 - DNA, 20 bp, Artificial Sequence
  Synthesized Sequence
tgacatattttgggcactct

SEQ ID NO: 30 - DNA, 20 bp, Artificial Sequence
  Synthesized Sequence
gtaaccatcctcaatttggt

SEQ ID NO: 31 - DNA, 20 bp, Artificial Sequence
  Synthesized Sequence
ggatgggatgtttcttagtc

SEQ ID NO: 32 - DNA, 20 bp, Artificial Sequence
  Synthesized Sequence
ctccaaatagacctctgtat

SEQ ID NO: 33 - DNA, 20 bp, Artificial Sequence
  Synthesized Sequence
cccctcaataaaaccagcaa

SEQ ID NO: 34 - DNA, 20 bp, Artificial Sequence
  Synthesized Sequence
aaccataccatccatctatc

SEQ ID NO: 35 - DNA, 20 bp, Artificial Sequence
  Synthesized Sequence
ttttttgatccgctgcatag

SEQ ID NO: 36 - DNA, 20 bp, Artificial Sequence
  Synthesized Sequence
tttgtaatcccgttaatggc

SEQ ID NO: 37 - DNA, 20 bp, Artificial Sequence
  Synthesized Sequence
ctcgataacagagttcacct

SEQ ID NO: 38 - DNA, 20 bp, Artificial Sequence
  Synthesized Sequence
tgtccaaatgtccagaaacc

SEQ ID NO: 39 - DNA, 20 bp, Artificial Sequence
  Synthesized Sequence
ggctttttactttctcgtac

SEQ ID NO: 40 - DNA, 20 bp, Artificial Sequence
  Synthesized Sequence
tccgatttctttggcattat

SEQ ID NO: 41 - DNA, 20 bp, Artificial Sequence
  Synthesized Sequence
tcattgtcacacttgtggta

SEQ ID NO: 42 - DNA, 20 bp, Artificial Sequence
  Synthesized Sequence
aagtcccatttcttacactt

SEQ ID NO: 43 - DNA, 20 bp, Artificial Sequence
  Synthesized Sequence
ctatcttttccctgttcaac

SEQ ID NO: 44 - DNA, 20 bp, Artificial Sequence
  Synthesized Sequence
cccattgattccaatttcac

SEQ ID NO: 45 - DNA, 20 bp, Artificial Sequence
  Synthesized Sequence
tggcgacagttgagtagatc

SEQ ID NO: 46 - DNA, 20 bp, Artificial Sequence
  Synthesized Sequence
gagaccaaaagcaccagtga

SEQ ID NO: 47 - DNA, 20 bp, Artificial Sequence
  Synthesized Sequence
acatccagaaactgattgcc

SEQ ID NO: 48 - DNA, 20 bp, Artificial Sequence
  Synthesized Sequence
atgcatattctgcactgcaa

SEQ ID NO: 49 - DNA, 22 bp, Artificial Sequence
  Synthesized Sequence
atcagacatgagaacagaatgg

SEQ ID NO: 50 - DNA, 23 bp, Artificial Sequence
  Synthesized Sequence
tgcctggcctgactagcaatatc

SEQ ID NO: 51 - DNA, 25 bp, Artificial Sequence
  Synthesized Sequence
cgaggtcgaaacgcctatcagaaac

SEQ ID NO: 52 - DNA, 26 bp, Artificial Sequence
  Synthesized Sequence
ccaatgatatttgctgcaatgacgag

SEQ ID NO: 53 - DNA, 21 bp, Artificial Sequence
  Synthesized Sequence
tggaaagcaaatagtggagcg

SEQ ID NO: 54 - DNA, 22 bp, Artificial Sequence
  Synthesized Sequence
gtagcgcgatgcaggtacagag

SEQ ID NO: 55 - DNA, 25 bp, Artificial Sequence
  Synthesized Sequence
caagctttcaggacatactgatgag

SEQ ID NO: 56 - DNA, 24 bp, Artificial Sequence
  Synthesized Sequence
cttctccaagcgaatctctgtaga

SEQ ID NO: 57 - DNA, 21 bp, Artificial Sequence
  Synthesized Sequence
tctatttggagccattgctgg

SEQ ID NO: 58 - DNA, 20 bp, Artificial Sequence
  Synthesized Sequence
tgcttttttgatccgctgca

SEQ ID NO: 59 - DNA, 20 bp, Artificial Sequence
  Synthesized Sequence
gtaacccgttgaaccccatt

SEQ ID NO: 60 - DNA, 20 bp, Artificial Sequence
  Synthesized Sequence
ccatccaatcggtagtagcg

SEQ ID NO: 61 - DNA, 21 bp, Artificial Sequence
  Synthesized Sequence
ccgcgagaagatgacccagat

SEQ ID NO: 62 - DNA, 25 bp, Artificial Sequence
  Synthesized Sequence
cgttggcacagcctggatagcaacg

SEQ ID NO: 63 - DNA, 21 bp, Artificial Sequence
  Synthesized Sequence
ggaattggaaccctgtttggc

SEQ ID NO: 64 - DNA, 23 bp, Artificial Sequence
  Synthesized Sequence
gtctctgattcgcatgtaaaggt

SEQ ID NO: 65 - DNA, 19 bp, Artificial Sequence
  Synthesized Sequence
gcccagcctacagacagtg

SEQ ID NO: 66 - DNA, 22 bp, Artificial Sequence
  Synthesized Sequence
gcagcaagagatccaatgatgg

SEQ ID NO: 67 - DNA, 20 bp, Artificial Sequence
  Synthesized Sequence
tctgggcatcccaagttgac

SEQ ID NO: 68 - DNA, 21 bp, Artificial Sequence
  Synthesized Sequence
tccgccttttgatctccgatt

SEQ ID NO: 69 - DNA, 23 bp, Artificial Sequence
  Synthesized Sequence
ggagaaaactgacttcgaggaat

SEQ ID NO: 70 - DNA, 21 bp, Artificial Sequence
  Synthesized Sequence
tccagacgtggagtagggtta

SEQ ID NO: 71 - DNA, 21 bp, Artificial Sequence
  Synthesized Sequence
ctttgagcatccgtcagaagt

SEQ ID NO: 72 - DNA, 22 bp, Artificial Sequence
  Synthesized Sequence
agtgtgacacatcaccagtaca

SEQ ID NO: 73 - RNA, 21 nt, Artificial Sequence
  Synthesized Sequence
gcuuccaucuuuugcaucauu

SEQ ID NO: 74 - DNA, 25 bp, Artificial Sequence
  Synthesized Sequence
caccgtgcttatggccattctcacg

SEQ ID NO: 75 - DNA, 25 bp, Artificial Sequence
  Synthesized Sequence
aaaccgtgagaatggccataagcac